SEQUENCE LISTING (1 sequence)

SEQ ID NO: 1 - protein, 10 aa, Artificial Sequence
  linker
GSAGSAAGSG

(19) United States
(12) Patent Application Publication
(10) Pub. No.: US 2010/0015615 A1
(43) Pub. Date: Jan. 21, 2010

(54) Identification and Isolation of Adult Stem Cells and Related Methods of Use

(75) Inventors: Robert Benezra, Hampton Bays, NY (US); Hyung-Song Nam, New York, NY (US)

Correspondence Address: Larson & Anderson, LLC;re: MSK, P. O. BOX 4928, DILLON, CO, 80435-4928, US

(73) Assignee: SLOAN-KETTERING INSTITUTE FOR CANCER RESEARCH, New York, NY (US)

(21) Appl. No.: 12/433,707

(22) Filed: Apr. 30, 2009

Related U.S. Application Data
(60) Provisional application No. 61/049,420, filed on Apr. 30, 2008.

(51) Int. Cl. C12Q 1/68; C12N 5/00

(52) U.S. Cl. 435/6; 435/325

(57) ABSTRACT

Inhibitor of DNA Binding-1 (Id-1) is a marker protein found in stem cells, including adult stem cells, which can be used an indicator of the “stem-ness” of the cells. This allows Id1 expression to be used in a method for identifying cells as potential stem cells involving the step of screening the cells for expression of Id1; and a method for isolating cells as potential stem cells comprising the step of separating cells that express Id1 from cells that do not. Expression of GFAP can be used as a secondary screen to isolate rare B1 type adult neuronal stem cells.

### STATEMENT OF RELATED APPLICATIONS

This application claims the benefit of U.S. Provisional Application No. 61/049,420, filed Apr. 30, 2008, which application is incorporated herein by reference in its entirety.

### BACKGROUND OF THE INVENTION

This application relates to identification and isolation of stem cells, particularly adult stem cells.

Stem cells are unspecialized (undifferentiated) cells. They retain the ability to divide throughout life and give rise to both new stem cells and to more differentiated/specialized cells which can take the place of cells that die or are lost. Thus, stem cells contribute to the body's ability to renew and repair its tissues, because unlike mature (differentiated) cells, they are not permanently committed to their fate. Stem cells are recognized as being “multipotent” or “pluripotent”, i.e. as having the ability to differentiate into more than one type of specialized mature cell. “Adult stem cells” are cells with these characteristics that are derived from non-embryonic sources. This can include neonates, older individuals, and umbilical cord blood. Other terms for “adult stem cells” include tissue stem cells, somatic stem cells and post-natal stem cells.

Adult stem cells may arise from many different tissue types. Thus, studies have identified bone marrow stem cells, peripheral blood stem cell, neuronal stem cells, hNT cells, muscle stem cells, liver stem cells, pancreatic stem cells, corneal limbal stem cells, mammary stem cells, salivary gland stem cells, stomach stem cells, skin stem cells, tendon stem cells, synovial membrane stem cells, heart stem cells, cartilage stem cells, thymic progenitor stem cells, dental pulp stem cells, adipose derived stem cells, umbilical cord blood and mesenchyme stem cells, amniotic stem cells, mesangioblasts, and colon stem cells. Because many adult stem cells are multipotent but not pluripotent, exploitation of adult stem cells may depend on the ability to readily identify and isolate stem cells of different types. This will facilitate research on what characteristics are essential to the “stemness” of the cells, and also provide for better isolation of cells for potential therapeutic applications.

Identification of cells as stem cells typically relies on the use of cell surface markers or cellular differentiation (CD) antigens as indicators of the genomic activity related to a particular differentiation state, or the absence of indicators of more differentiation (such as expression of specialized enzymes). Among the surface markers that have been proposed for this purpose are CD117 (c-kit) for bone marrow stem cells, CD133 (AC133-2) which is found on multiple stem cell populations, and C1qRp, the receptor for complement molecule C1q, which is found on a subset of human CD34^{±} human stem cells form bone marrow and umbilical cord blood. It has not been found, however, that there is one cell surface marker that can be used as an indicator of stem-ness for cells of multiple tissue types.

### SUMMARY OF THE INVENTION

It has now been found by the present inventors that Inhibitor of DNA Binding-1 (Id-1) is a marker protein found in stem cells, including adult stem cells, which can be used an indicator of the “stem-ness” of the cells. Thus, in accordance with present invention, there is provided:
(1) a method for identifying cells as potential stem cells comprising the step of screening the cells for expression of Id1; and
(2) a method for isolating cells as potential stem cells comprising the step of separating cells that express Id1 from cells that do not.

In specific embodiments of these inventions, the cells are potentially adult stem cells derived from neuronal tissue, olfactory epithelium, colon, bone marrow (hematopoietic stem cells), breast epithelium, eye, and germline tissue.

The invention further provides for the use of such cells in a variety of applications including:
(1) identification of promoters or inhibitors of differentiation for the specific tissue type;
(2) study of other elements contributing to “stem-ness”; and
(3) in vitro growth of tissues for therapeutic uses;

The cells also have potential therapeutic application as the understanding of stem cell usage in vivo increases.

### BRIEF DESCRIPTION OF THE DRAWINGS

FIG. 1 diagrammatically shows the construction of an inbred knock-in mouse strain which expresses an Id1-VenusYFP fusion protein. In mice carrying the Id1-Venus allele, a fusion protein of Id1 and Venus yellow fluorescent protein linked by a 10-amino acid flexible linker (GSAGSAAGSG, SEQ ID No: 1) is expressed from the endogenous Id1 locus. In vitro studies demonstrated that the fusion protein retains the activity of the wildtype Id1 protein

FIGS. 2A and B show the results of flow cytometric measurements and immunofluorescence experiments on Id1 expression in NS cell cultures.

FIGS. 3A-C shows results of clonal neurosphere assays on Id1⁺ and Id1⁻ cells demonstrating that Id genes are necessary for self-renewal, a characteristic of stem cell identity, but dispensable for proliferation. A. clonal neurosphere assays, FACsorted Id1_{high} cells formed larger (3B) and more numerous neurospheres (3A) that could be passaged (3C) while Id1_{low} cells did not. Unpaired two-tailed Student's t-test. DIV=days in vitro. Scale bar=50 microns.

FIG. 4 shows the results of flow cytometric cell cycle kinetics measurements on Id1⁺ and Id1⁻ NS cells.

FIG. 5 shows results of quantitative RT-PCR which demonstrated high and low Id1 mRNA levels in the Id1_{high} and Id₁_{low} populations relative to control unfractionated cells. Furthermore, Affymetrix microarray analyses supported stem-like and neuroblast-like identities of the Id1_{high} and Id1_{low} NS cells, respectively. The data are summarized in Venn diagrams. At least 1,325 probes were differentially expressed in the two populations. Id2, Id3, and Id4 were not differentially expressed. Notable over-expressed transcripts in the Id1_{high} cells were Id1, signaling molecules and receptors, JAK-STAT, MAPK, and Notch pathway effectors, homeobox transcription factors, zinc finger transcription factors, POU domain transcription factors, Krüppel-like factors, and finally, cyclin, and cyclin-dependent kinase inhibitors. The expression of transcripts associated with neural or embryonic stem cell self-renewal and/or pluripotency was consistent with a stem cell identity of the Id1_{high} cells

FIG. 6 shows the targeting strategy for the Id1-floxed mouse strain. Adult Id1_{-/-} and Id3_{-/-} mice are viable and fertile. However, deletion of both Id1 and Id3 causes embryonic lethality at ˜12.5 days of embryogenesis due to neurogenesis, vascular, and cardiac defects (Fraidenraich et al., 2004; Lyden et al., 1999), and precludes experiments with adult stem cells. Thus, we developed a floxed Id1 allele for conditional deletion. In mice carrying the Id1-floxed allele, the entire Id1 gene is flanked by loxP sites and can be excised by cre-recombinase

FIG. 7 shows results of clonal neurosphere assay. In bulk neurosphere assays, Id1 and Id3, but not Id1 alone, were required for secondary neurosphere formation. ˜50% efficiency nucleofection of cre cDNA resulted in ˜50% reduction of secondary neurosphere cells. Neurosphere cells from Id1_{-/-} and Id3_{-/-} mice showed unimpaired secondary sphere formation.

FIG. 8 shows types of self-renewal and proliferation.

FIG. 9 shows actual data versus predicted behavior for the self-renewal models of FIG. 8.

FIGS. 10A-C show Flow cytometric immunophenotyping of the anterior subventricular cells from Id1 V/V mic which confirmed and quantified the rare population of the Id1-expressing GFAP₊ astrocytes (10A as well as the Mash1₊ cells (10B). PSA-NCAM₊ cells did not express Id1 (10C).

FIGS. 11A and B illustrate that adherent SVZ neural stem/progenitor cultures (NS cells) are molecularly heterogeneous via cytometric measurements of the Id1-Venus levels in early passage NS cells, with at least two distinct populations.

FIG. 12 shows the experimental procedure for flow cytometric analyses of subventricular cells.

FIGS. 13A and 13B show that subventricular endothelial cells also express Id1. Flow cytometric phenotyping of CD31 and Id1-Venus in dissociated Id1_{V/V} anterior SVZ cells demonstrated and quantitated the subventricular CD31+Id1_{high} endothelial cells.

FIG. 14 shows the targeting strategy used to create the Id1-IRES-creERT2 mouse strain. In mice carrying the Id1-IRES-creERT2 allele, tamoxifen-inducible cre recombinase creERT2 is expressed from a bicistronic messenger RNA by means of an EMCV internal ribosome entry site.

FIG. 15 shows the targeting strategy used to create the ROSA26-StLa mouse strain. In mice carrying the targeted Stop-tauLacZ (StLa) allele, cre+ cells and their progeny express the tauLacZ reporter gene driven by the CAG constitutively active CMV enhancer-chicken β-actin hybrid promoter (Niwa et al., 1991). The StLa allele was created to enable unambiguous and morphological identification in the adult brain. The R26R reporter allele (Soriano, 1999) is widely used for lineage tracing studies. However, in the adult brain, the expression level of the β-gal protein driven by the endogenous ROSA26 promoter is low and can be difficult to detect unambiguously. In contrast, expression level in the adult brain of the tauLacZ-encoded tau-β-gal fusion protein driven by the CAG promoter is comparatively higher than the expression level of β-gal from the ROSA26 promoter, enabling unambiguous labeling. Though X-gal staining reveals intense blue precipitate in the entire cell, regardless of cell type, immunofluorescence analyses reveal distinct tau-β-gal localization in different cell types. In subventricular astrocytic cells, tau-β-gal labels cytoplasm. In neuronal cells, tau-β-gal labels cell body and neuronal processes. In oligodendrocytic cells, tau-β-gal labels processes.

FIG. 16 shows a fate mapping strategy. Various reporter alleles were used to reveal all or subsets of Id1-expressing cells and their progeny. StLa and R26_{LSL-YFP} alleles utilize the nearly ubiquitously-expressed CAG and Gt(ROSA26) promoters. The GFAP::LSL-GFP transgene utilizes the astrocyte-specific ˜2.2 kb Gfa2 human GFAP promoter. Tau_{LSL-mGFP-IRES-nLacZ} allele utilizes the neuron-specific endogenous Tau (Mapt) promoter. With all reporter alleles, cre-mediated recombinatorial excision of the “stop cassette” enables reporter expression in cell types in which the reporter gene promoter is active, i.e., all, astrocytic, or neuronal cell types. As neither DCX+ neuroblasts nor NeuN+ neurons express Id1, no labeled cells are expected at the three day time point in Id1_{IRES-creERT2}; Tau_{LSL-mGFP-IRES-nLacZ} mice.

FIG. 17 shows that subventricular GFAP+Id1_{high} B1 type astrocytes are neurogenic stem cells and in particular shows that the neuronal output increased over time in Id1_{IRES-creERT2}; Tau_{LSL-GFP-IRES-nLacZ} mice, in which the neuronal progeny of the Id1_{high} cells are specifically labeled by genetic means.

FIG. 18 evidences that cultured subventricular Id1_{high} stem cells can self-renew asymmetrically. In low density cultures, purified subventricular Id1_{high} stem cells (solid line) generated Id1_{high} and Id1ᵢₙₜ cells whereas purified Id1_{low} neuroblasts (dashed line) generated only Id1_{low} cells. Id1 gate and the percentage of Id1_{high} and Id1_{low} cells are shown.

FIG. 19 provides further evidence that cultured subventricular Id1_{high} stem cells can self-renew asymmetrically. Consistent with asymmetric self-renewal of the Id1_{high} stem cells, the frequency of the Id1_{high} cells gradually decreased during serial passages.

### DETAILED DESCRIPTION OF THE INVENTION

The present application relates to a method for identifying cells as potential stem cells comprising the step of screening the cells for expression of Id1; and a method for isolating cells as potential stem cells comprising the step of separating cells that express Id1 from cells that do not. The invention further relates to the use of such cells in a variety of application.

Definitions

While the terms in this application are generally used in a manner consistent with usage in the art, the following definitions are provided for clarity:

“stem cell” refers to a cell that retains the ability to divide throughout life and give rise to both new stem cells and to more differentiated/specialized cells.

“adult stem cell” refers to stem cells derived from (either directly or as a source of a cell line) from a non-embryonic source.

“potential stem cell” refers to cells that may be stem cells but which may need further separation from non-stem cells based on secondary markers specific to a given tissue type of stem cell to provide fully isolated stem cells.

“Id protein” refers to an Inhibitor of DNA-Binding protein which are known genes that encode transcriptional regulators that function as naturally occurring dominant-negative antagonists of the basic-helix-loop-helix transcription factors. (Benezra, 1990. Id proteins are known in the art for numerous species. Individual Id proteins have been labeled as Id1, Id2, Id3 and Id4. Amino acid and gene sequences for these proteins are known in the art, and available through GenBank, for example with the following accession numbers for Id1 and Id3 proteins.

For Id1:

| Accession No. | Species |
|---|---|
| U57645 | Human |
| BC000613 | Human |
| BC012420 | Human |
| BG546814 | Human |
| D13889 | Human |
| D13890 | Human |
| S78825 | Human |
| S78986 | Human |
| X77956 | Human |
| AK008264 | Mouse |
| BC025073 | Mouse |
| M31885 | Mouse |
| U43884 | Mouse |
| AI073271 | Rat |
| D10862 | Rat |
| L23148 | Rat |
| M86708 | Rat |

For Id3:

| Accession NO. | Species |
|---|---|
| X73428 | Human |
| BC003107 | Human |
| BM921819 | Human |
| D28449 | Human |
| X66924 | Human |
| X69111 | Human |
| A17546 | Human |
| A17548 | Human |
| M60524 | Mouse |
| AI173823 | Mouse |
| AK002820 | Mouse |
| AK014306 | Mouse |
| AK031499 | Mouse |
| M60523 | Mouse |
| AF000942 | Rat |
| D10864 | Rat |

Id genes are necessary and sufficient for context-dependent regulation of the cell cycle status and differentiation. (Perk, 2005) Moreover, Id genes are sufficient for the maintenance of murine embryonic stem cell self-renewal and pluripotency in the absence of bone morphogenetic protein (Ying, 2003) and Id1 is necessary for long-term hematopoietic stem cell self-renewal. (Jankovic, 2007; Perry, 2007).

Experimental Identification of Id1 as a Stem Cell Marker

This invention is premised on Applicants' discovery that Id1 is a non-tissue specific stem cell marker. This determination was made through a series of experiments using the following materials and methods.

Cryostat sections from adult (6-weeks) mice were examined by immunofluorescence and confocal microscopy. Gene targeted mice were generated with C57BL/6 embryonic stem cells and targeting vectors recombineered from C57BL/6J bacterial artificial chromosome. Microdissected and papain-dissociated adult mice SVZ cells were labeled with antibodies and measured on a 2-laser FACSCalibur. Id1+ cells were sorted from papain-dissociated adult SVZ cells with a MoFlo sorter and cultured in serum-free media with FGF-2 and EGF in low-binding plates. For adherent culture, SVZ cells were cultured in poly-ornithine and laminin coated plates in the same media. Array data will be deposited in NCBI GEO database.

Gene Targeted Mice

Targeting vectors were recombineered (Liu, 2003; Warming, 2005)) from RPCI-23 C57BL/6J bacterial artificial chromosome clones (CHORI). Bruce4 C57BL/6 ES cells26 or CY2.4 B6(Cg)-Tyrc-2J/J ES cells (C. Yang, The Rockefeller University Gene Targeting Core Facility, unpublished work) were electroporated and G418-resistant clones screened by Southern blots. Targeted clones were confirmed by additional Southern blots, karyotyped (M. Leversha, Molecular Cytogenetics Core), and injected into FVB, B6(Cg)-Tyrc-2J/J, or B6 blastocysts. Neo cassette was excised in vivo with B6 ACTB::FLPE mice27 and confirmed by Southern blots. Animal work were in accordance with Memorial Sloan-Kettering Cancer Center's Institutional Animal Care and Use Committee policies.

Immunofluorescence and Microscopy

Ten μm cryostat sections from 6-week-old mice were analyzed. Commercially-available antibodies were Nestin (BD Biosciences), GFAP (Zymed), PSA-NCAM (Chemicon), CD15 (BD Biosciences), Tuj1 (Covance Research Products), beta-galactosidase (Abcam), and Alexa488, 594, or 647 secondary antibodies (Invitrogen). Samples and cells were imaged with a confocal microscope (Leica) or an inverted microscope (Zeiss).

Flow Cytometry

SVZ was microdissected from 1 mm coronal slices from 6-week-old mice (Lois 1993). Cells dissociated with papain (Worthington Biochemical Corporation) were labeled with indicated antibodies. For live/dead discrimination, either propidium iodide (Invitrogen) or 7-AAD (Calbiochem) was employed. For phenotyping, ˜20,000 events were acquired with a 2-laser FACSCalibur (BD). Cells were sorted with a MoFlo sorter (DakoCytomation). For BrdU detection, cells were pulsed for 1 h with 10 μM BrdU (Sigma), and BrdU-APC Kit (BD Biosciences) was employed, with minor modifications. Cell cycle parameters were calculated with formulae 1, 2b, 7, 8, 10, and 11 in N. H. Terry and R. A. White, Nature protocols 1 (2), 859 (2006).

Tamoxifen Induction

6 mg of tamoxifen (Sigma) dissolved in 10% ethanol and 90% corn oil was injected intraperitoneally, once per day, for two consecutive days. Mice were >8 weeks old.

Cell Culture and Neurosphere Assays

Adult SVZ cells were cultured in Neurobasal-A (Invitrogen), B27 without vitamin A (Invitrogen), GlutaMax-1 (Invitrogen), Pen/Strep (Invitrogen), rmEGF (R&D), rmFGF-2 (R&D), and heparin (Sigma). For adherent culture, cells were plated in poly-ornithine (Sigma) and laminin (Sigma) coated plates (Falcon). Cells were dissociated with Accutase (Invitrogen) and passaged every ˜2-3 days. For clonal primary neurosphere assay, 200 Id1+ or Id1− cells were FAC sorted directly into each well of a 96-well low-binding plate (Corning) with fresh media and cultured for 7-10 days. For secondary sphere self-renewal assay, spheres in each well were dissociated with Accutase, washed twice with phosphate-buffered saline, and replated into another well with fresh media. Number of secondary spheres was counted 10 days post-plating.

For self-renewal assay, cells were nucleofected (Mouse Neural Stem Cell Kit, Amaxa) with pCAG-cre-IRES-EGFP plasmid (Woodhead, 2006), ˜50% GFP+, measured by flow cytometry), and 0.5 million cells plated per well of a 6-well low-binding plates. Twelve days postplating, neurospheres were trypsinized and counted with a Coulter counter (Beckman Coulter).

Microarray and Quantitative RT-PCR

RNA was purified from FAC sorted cells with RNeasy Micro Kit (Qiagen). Labeled cRNA was hybridized to MOE 430 2.0 arrays (Affymetrix). Data were analyzed in GeneSpring GX 7.3.1 (Agilent). Array data will be deposited in NCBI GEO database. CT values of Id1 and GAPDH TaqMan assays (Applied Biosystems) from triplicate samples were normalized and CT value calculated.

Cell Pair Assay

Id1^{V/V} NS cells were FAC sorted and plated on poly-ornithine and laminin coated tissue culture slides (Nunc) at clonal density and confirmed at ˜8 h post-plating. At ˜72 h postplating, cells were fixed with 4% paraformaldehyde in phosphate-buffered saline and imaged with a confocal microscope (Leica).

Using these methods, we examined whether Id1 is expressed in putative neural stem/progenitor cells. In the SVZ, antibodies against intermediate filament proteins nestin or glial fibrillary acidic protein (GFAP) label astrocyte-like “B” cells, and antibodies against polysialylated neural cell adhesion molecule (PSA-NCAM) or III-tubulin (Tuj1) label migratory neuroblast cells (Doetsch, 1997). Two- or three-color immunofluorescence analyses of adult mouse brain sections with the rabbit monoclonal anti-Id1 antibody (Perk, 2006) and additional antibodies revealed Id1 in a subset of nestin+ GFAP+ astrocyte-like cells in the adult murine SVZ, but not in PSA-NCAM+ or III-tubulin+ differentiated neuroblast cells. CD31+ endothelial cells were also Id1+. Co-localization of Id1 and GFAP was confirmed in dissociated SVZ cells.

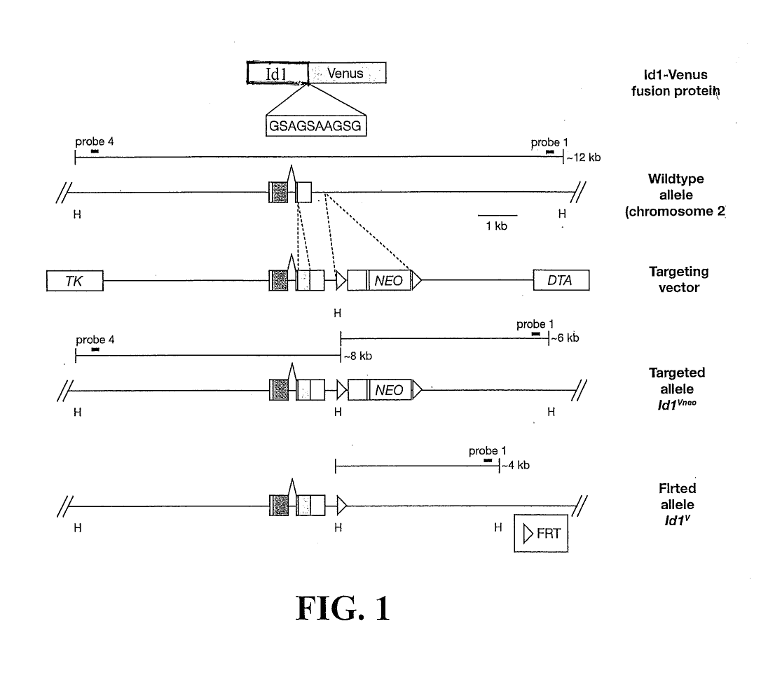

We further confirmed the immunohistological findings using a novel inbred knock-in mouse strain which expresses an Id1-VenusYFP fusion protein (FIG. 1). Preliminary experiments had established that Id1-Venus fusion protein maintains Id1 protein activity and that Id1-Venus+ cells are Id1+. Flow cytometric phenotyping of the Id1^{V/V} SVZ cells indicated 3.1±0.64% of the dissected SVZ cells were Id1+ (n=14, mean±standard deviation). ˜6% of GFAP+ cells were Id1+ (16% of Id1+ cells); ˜9% of CD15+ cells were Id1+ (˜43% of Id1+ cells); and ˜52% of CD31+ cells were Id1+ (˜44% of Id1+ cells). PSA-NCAM and Id1 did not co-localize. Thus, most, if not all, of the SVZ Id1+ cells were either CD15+ or CD31+. We note that the percentage of GFAP+ and PSA-NCAM+ cells is consistent with data in A. Capela, S. Temple, Neuron 35, 865 (Aug. 29, 2002).; however, the percentage of CD15+ cells is higher in our experiments, likely due to dissociation protocol differences. These experiments suggest that Id1 defines the neural stem cell subset of nestin+ GFAP+ astrocyte-like cells. Thus, we further investigated the functional capacity of the Id1+ subventricular zone cells. We assayed stem cell activity of the Id1+ fraction ex vivo by neurosphere assay as described in B. A. Reynolds, R. L. Rietze, Nat Methods 2, 333 (May, 2005). Id1+ cells purified from the dissociated SVZ cells formed neurospheres that self-renewed and differentiated into beta-III-tubulin+ neurons and GFAP+ astrocytes, showing the two hallmarks of neural stem cell activity.

To determine whether the Id1+ cells give rise to more differentiated progeny in vivo, we performed lineage tracing experiments using Id1^{IRES-creERT2} knock-in mice. At seven days after tamoxifen induced labeling of Id1+ cells and their progeny cells in Id1^{IRES-creERT2}; R26R compound heterozygous mice, some of the labeled (beta-galactosidase+) cells were GFAP+, consistent with the immunofluorescence analyses. In addition, although GFAP− Id1+ PSA-NCAM+ cells were never observed in the immunofluorescence analyses, seven days post-tamoxifen induction, some of the beta-gal+ cells were PSA-NCAM+, indicating a more differentiated identity of the labeled cells. At an earlier time point of three days after tamoxifen induction, beta-gal+ cells were not detected (≦1 cell per SVZ), possibly due to low creERT2 efficiency and low ROSA26 transcriptional activity in the adult brain. We conclude that the Id1+ cells are capable of producing more differentiated progeny in vivo.

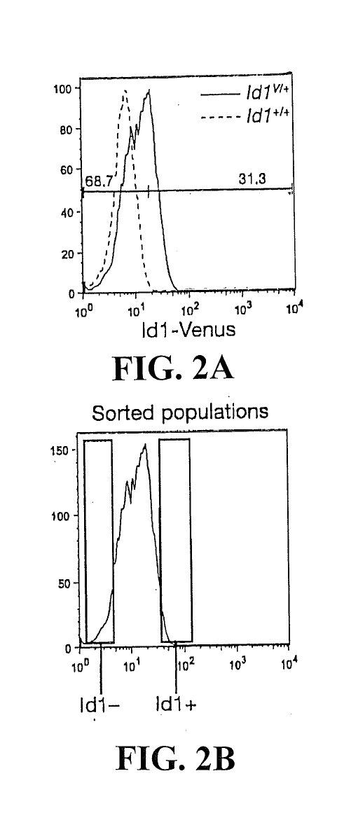

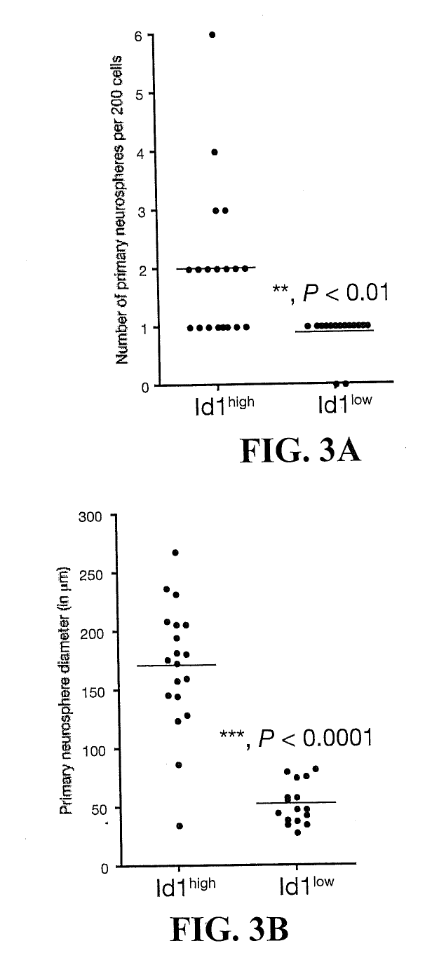

We also assayed the stem cell activity of the Id1+ fraction in early and later passage Id1^{v/+} and Id1^{V/V} adult SVZ adherent NS cell cultures (L. Conti et al., PLoS Biol 3, e283 (September, 2005)); flow cytometric measurements and immunofluorescence experiments had indicated Id1 expression in NS cell cultures is heterogeneous (FIGS. 2A and B). When Id1+ and Id1− cells were purified and compared in clonal neurosphere assays, Id1+ cells generated more and larger neurospheres (FIG. 3, neurosphere number, 2-fold increase, n=19, P<0.01; neurosphere size, 3.5-fold increase, n=19, P<0.0001; unpaired two-tailed Student's t-test); upon passaging, only Id1+ cell-derived spheres self-renewed (n=18), indicative of stem cell activity. Id1− cell-derived spheres did not self-renew (FIG. 3); however, the Id1− cells were morphologically undifferentiated and proliferative, and when plated as adherent monolayer, the Id1− cells proliferated more rapidly than Id1+ cells. These results indicate that Id1− cells may be anchorage-dependent transit amplifying cells, and furthermore, that Id1 is not required for proliferation per se.

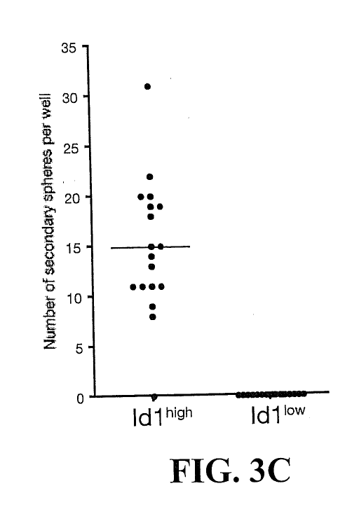

To directly measure the cell cycle kinetics of the Id1+ and Id1− cells, NS cells were pulsed with the thymidine analog bromodeoxyuridine (BrdU), chased, then sorted. Flow cytometric cell cycle kinetics measurements confirmed the non-equivalence of the two populations, and further, indicated that while a greater fraction of the Id1+ cells were in S-phase (% BrdU+, Id1+: 44% vs. Id1−: 17%, FIG. 4), these cells transit more slowly through the cell cycle than the Id1− cells (at 12 h post-BrdU, 2n population of BrdU+ cells, Id1+: 20% vs. Id1−: 48%, FIG. 4), implying a delay in G2/M-phase progression. The S-phase duration of the Id1+ and Id1− cells were ˜30 h and ˜17 h, respectively, and the doubling time of the Id1+ and Id1− cells were ˜64 h and ˜36 h, respectively.

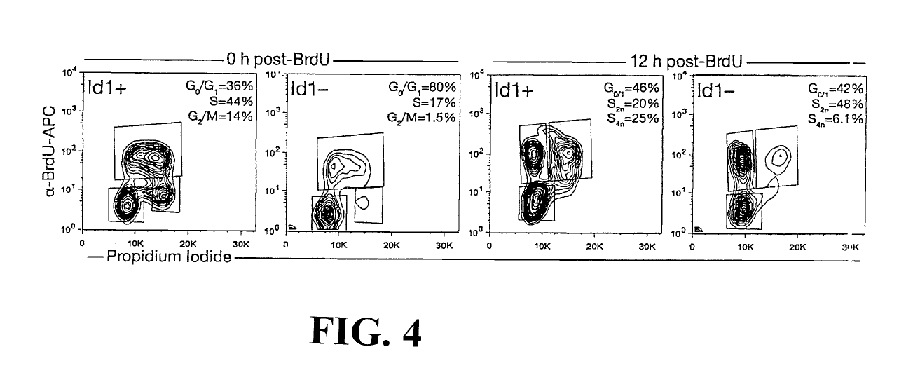

The experiments described indicated that the Id1 +and Id1− cells were functionally distinct-asymmetric-cell types. We thus examined the molecular profile of the Id1+ and Id1− cells for transcripts associated with each cell type. Quantitative RT-PCR had confirmed differential Id1 mRNA expression in the two cell types (FIG. 5). At least 1,325 probes were differentially expressed. Id2, Id3, and Id4 were unchanged in the Id1+ cells. Notable up-regulated transcripts in the Id1+ cells were Id1, signaling molecules and receptors, JAK-STAT, MAPK, and Notch pathway effectors, homeobox transcription factors, zinc finger transcription factors, POU domain transcription factors, Krüppel-like factors, and finally, cyclin, and cyclin-dependent kinase inhibitors. The expression of transcripts associated with neural or embryonic stem cell self-renewal and/or pluripotency suggested a stem cell identity of the Id1+ cells (Q. L. Ying, J. Nichols, I. Chambers, A. Smith, Cell 115, 281 (Oct. 31, 2003); A. Androutsellis-Theotokis et al., Nature 442, 823 (Aug. 17, 2006); S. W. Bruggeman, M. van Lohuizen, Cell Cycle 5, 1281 (June, 2006); L. S. Campos et al., Development 131, 3433 (July, 2004); K. Takahashi, S. Yamanaka, Cell 126, 663 (Aug. 25, 2006)). Furthermore, the expression of multiple homeobox transcription factors suggested that the Id1+ cells are a heterogeneous population of stem cells originating from multiple embryonic origins (F. T. Merkle, Z. Mirzadeh, A. Alvarez-Buylla, Science 317, 381 (Jul. 20, 2007); K. M. Young, M. Fogarty, N. Kessaris, W. D. Richardson, J Neurosci 27, 8286 (Aug. 1, 2007)). Notable up-regulated transcripts in the Id1− cells were Camk2a, CD24a, Elav14, Foxg1, Gad1, Mapt, Ntrk3, Sall3, Sox4, and Thy1. These suggested a more differentiated identity. However, up-regulation of some of these transcripts, suggestive of a neuroblast or neuronal identity, was surprising, considering that the Id1− cells were morphologically undifferentiated, proliferative, and capable of primary (but not secondary) neurosphere formation. Moreover, Dlx2, EGFR, and Olig2 transcripts, previously reported as “C” transit amplifying cell markers (F. Doetsch, L. Petreanu, I. Caille, J. M. Garcia-Verdugo, A. Alvarez-Buylla, Neuron 36, 1021 (Dec. 19, 2002); M. A. Hack et al., Nat Neurosci 8, 865 (July, 2005)), were unchanged in the Id1− cells. However, up-regulation of Foxg1, Sall3, and Sox4 suggested a transit amplifying cell identity of the Id1− cells: Foxg1 is required for forebrain development and timing of embryonic cortical progenitor neurogenesis (Q. Shen et al., Nat Neurosci 9, 743 (June, 2006); S. Xuan et al., Neuron 14, 1141 (June, 1995)); Sall3 is expressed in the embryonic neural structures and may be required for migration of neural progenitors (M. Barembaum, M. Bronner-Fraser, Neuron Glia Biol 1, 57 (February, 2004); T. Ott, M. Parrish, K. Bond, A. Schwaeger-Nickolenko, A. P. Monaghan, Mech Dev 101, 203 (March, 2001); M. Parrish et al., Mol Cell Biol 24, 7102 (August, 2004)); Sox4 is expressed in differentiating progenitor cells and may be important for neurogenesis (M. Bergsland, M. Werme, M. Malewicz, T. Perlmann, J. Muhr, Genes Dev 20, 3475 (Dec. 15, 2006); M. Cheung, M. Abu-Elmagd, H. Clevers, P. J. Scotting, Brain Res Mol Brain Res 79, 180 (Jun. 23, 2000)). We conclude Id1+ and Id1− cells are functionally and molecularly asymmetric stem and transit amplifying cells, respectively.

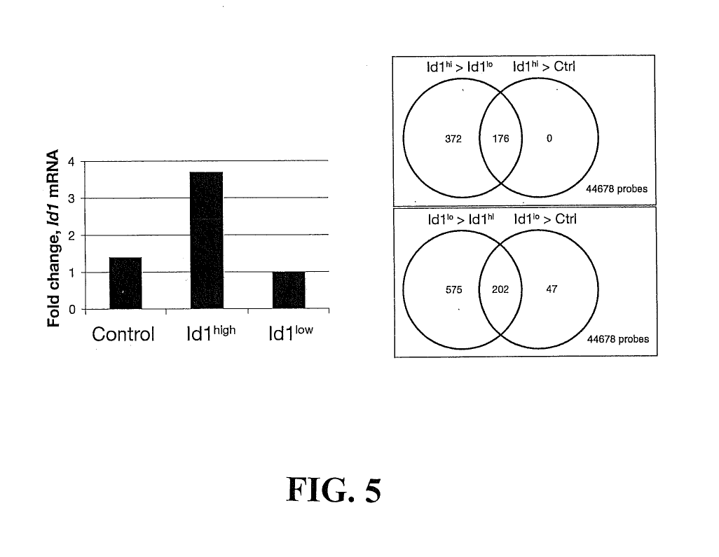

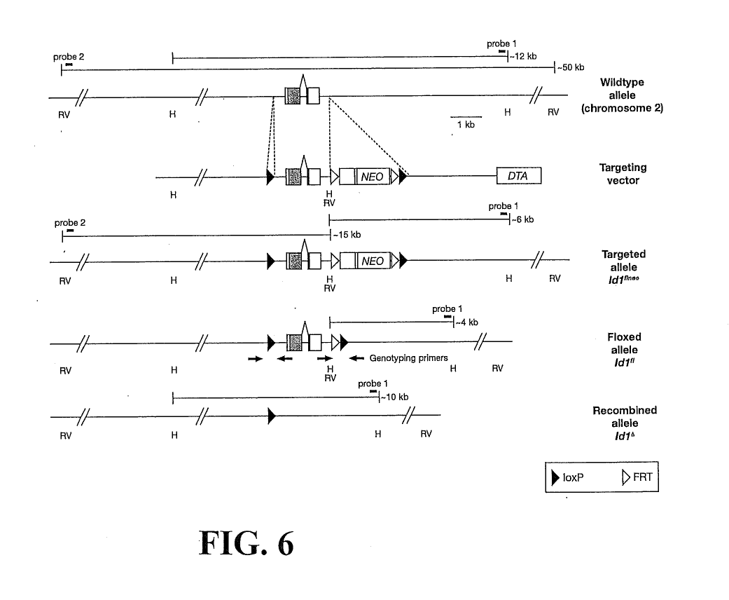

We then asked whether Id genes are required for stem cell self-renewal. Adult Id1 or Id3 null mice are viable and fertile, and neurosphere self-renewal is normal, suggesting redundancy or compensation. However, deletion of both Id1 and Id3 causes embryonic lethality at ˜12.5 days of embryogenesis due to neurogenesis, vascular, and cardiac defects (D. Fraidenraich et al., Science 306, 247 (Oct. 8, 2004); D. Lyden et al., Nature 401, 670 (Oct. 14, 1999)), and precludes experiments with adult stem cells. To circumvent the embryonic lethality of Id1;Id3 deletion, we generated a conditional Id1 allele (FIG. 6) and delivered cre recombinase cDNA into neurospheres. Ablation of Id1 and Id3, but not Id1 alone, was sufficient to reduce secondary neurosphere formation by ˜50% (FIG. 7); genomic PCR detected the unrecombined floxed allele in neurospheres that formed. The slight increase in cell number of Id1^{fl/fl};Id3^{+/+} neurospheres may be due to transient compensatory increase in Id3 expression. In summary, we conclude Id genes are required for stem cell self-renewal.

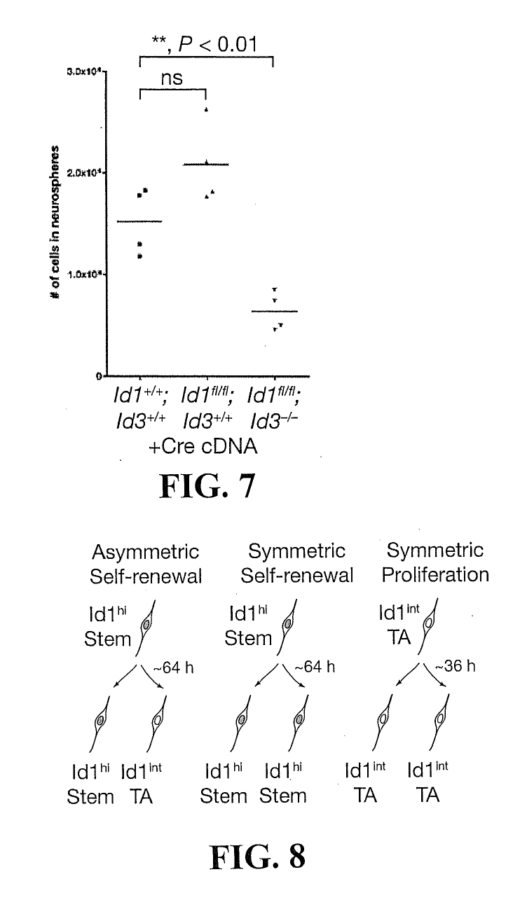

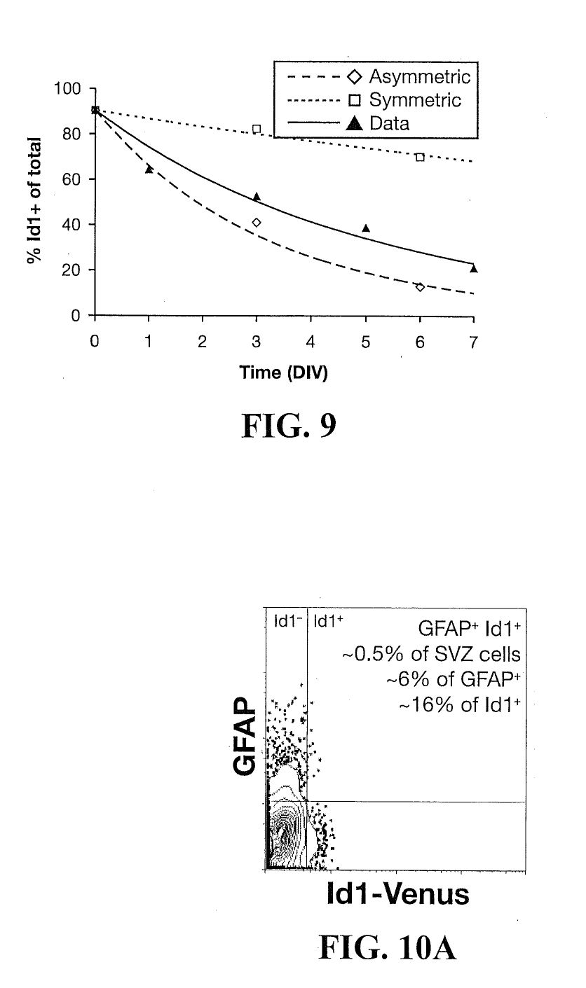

Finally, we tested the hypothesis that the progeny of Id1+ stem cells may differ in Id1 expression. We examined the progeny of the Id1+ stem and Id1− transit amplifying cells in vitro by culturing the purified cells individually and measuring the percentage of Id1+ and Id1− cells flow cytometrically. In a serum-free medium with FGF-2 and EGF, the Id1+ stem cells generated Id1+ and Id1− cells (as evidenced by the leftward shift of the histogram), but the Id1− transit amplifying cells generated only Id1− cells and did not generate Id1+ stem cells. This experiment indicated that, first, the Id1+ stem cell self-renewal generates a more differentiated cell type which differs in Id1 level; and second, the differentiation of Id1+ stem cell to Id1− transit amplifying cell is unidirectional and irreversible. Based on the observed kinetics of dilution of Id1+ cells in FIG. 4A, we calculated the percentage of Id1+ cells generated by asymmetric or symmetric self-renewal of the Id1+ stem cells and symmetric (and more rapid) proliferation of the contaminating Id1− transit amplifying cells (FIG. 8). Based on the model, the experimental measurements were consistent with the population kinetics of asymmetric self-renewal of the ˜30-50% Id1+ stem cells and symmetric proliferation of the contaminating Id1− transit amplifying cells (FIG. 9 and attached Model). We also examined the progeny of the Id1+ stem cells by cell pair assay (Q. Shen, W. Zhong, Y. N. Jan, S. Temple, Development 129, 4843 (October, 2002)). Id1+ stem cells generated Id1+ stem and Id1− transit amplifying cells. One prediction of the asymmetric self-renewal of the Id1+ stem cells is that the percentage of Id1+ cells would gradually decrease as a function of the cell division number. Indeed, comparisons of early and later passage cultures indicated gradual reduction of the percentage of the Id1+ stem cell. The early and later passage NS cells were morphologically identical. In contrast to a previous report (L. Conti et al., PLoS Biol 3, e283 (September, 2005)), we conclude that the majority of neural stem cells self-renew asymmetrically in vitro.

We have shown that the Id1+ cells are neural stem cells, that the Id genes are required for neural stem cell self-renewal, that the Id1+ neural stem cell progeny differ in Id1 expression, and that the percentage of Id1+ neural stem cells gradually decrease during culture. Thus, we conclude that Id1+ neural stem cells self-renew asymmetrically and that Id1 is a molecular determinant of the functional asymmetry in the adult neural stem cell progeny. Thus, asymmetric localization of transcription factors or regulators establishes the functional asymmetry between stem cells and their progeny in mammalian neurogenesis.

When similar experiments in other types of adult stem cells are conducted, similar results are obtained showing that Id1 expression functions as a marker of “stem-ness.” In particular, this result is obtained when experiments results are conducted in putative olfactory epithelium, colon, HSC, breast epithelium, eye, and germline stem cells.

Experimental Identification of Id1 and GFAP as Markers of B1 Type Adult Neural Stem Cells

The present invention is particularly applicable for the identification and isolation of the B type astrocytes which may be required for the generation of neuroblasts and neurons (Garcia et al., 2004). These cells, also referred to as “B1 type adult neural stem cells” are thought to be responsible for subventricular neurogenesis, but have proven difficult to isolate and characterize. The inventors have now found that selection of cells of neuronal origin that are GFAP-positive and Id1 positive provides long-lived, and relatively quiescent GFAP+ Id1_{high} astrocytes with morphology characteristic of B1 type astrocytes that self-renew and generate migratory neuroblasts that differentiate into olfactory bulb interneurons.

A Decreasing Gradient of Id1 Levels in the Subventricular Neurogenic Lineages

Preliminary diaminobenzidine immunohistochemistry and tyramide signal amplification (TSA) immunofluorescence analyses of Id1_{+/+} and Id1_{-/-} adult mouse brains with the highly specific rabbit monoclonal anti-Id1 antibody (Perk et al., 2006) revealed specific Id1 immunoreactivity in the parenchyma and in the subventricular region. At least two morphologically distinguishable types of Id1+ nuclei, round and elongated, were evident.

Further TSA immunofluorescence and confocal microscopic analyses with the anti-Id1 antibody and anti-GFAP antibody revealed predominantly round Id1 immunoreactivity in the GFAP+ subventricular region in the adult SVZ. Immunostaining of dissociated subventricular cells confirmed the presence of the Id1 and GFAP antigens in a single cell, indicating that some of the subventricular cells with the round Id1+ nuclei are GFAP+ astrocytes.

TSA immunofluorescence of Id1 and Mash1, a transit amplifying cell marker, revealed co-localization of the two nuclear antigens in some subventricular cells. However, Mash1 immunoreactivity was evident in only a minority of the Id1+ cells. Cells that were Id1+ Mash1−, as well as Id1− Mash1+ were evident. Interestingly, co-expression of Id1 and Mash1 appeared to be limited to cells expressing comparatively lower levels of Id1. Thus, a gradient of Id1 and Mash1 levels was apparent in the subventricular Id1-expressing cells, and the cells expressing lower levels of Id1, the Id1ᵢₙₜ cells, co-expressed Mash1, a marker of a more differentiated phenotype.

Consistent with a gradually decreasing gradient of Id1 levels along the subventricular neurogenic lineages, PSA-NCAM+ neuroblasts or NeuN+ neurons never expressed Id1. Moreover, S100b+ ependymal cells also never expressed Id1. Importantly, S100b+ parenchymal astrocytes were Id1−, suggesting subventricular astrocyte-specific Id1 expression.

Finally, the subventricular and parenchymal cells with elongated Id1+ nuclei were CD31+ endothelial cells. In the SVZ, the two morphologically distinguishable Id1+ cell types were often located nearby, suggesting that the GFAP+ Id1_{high} cells reside in or near the perivascular stem cell niche (Shen et al., 2008; Tavazoie et al., 2008).

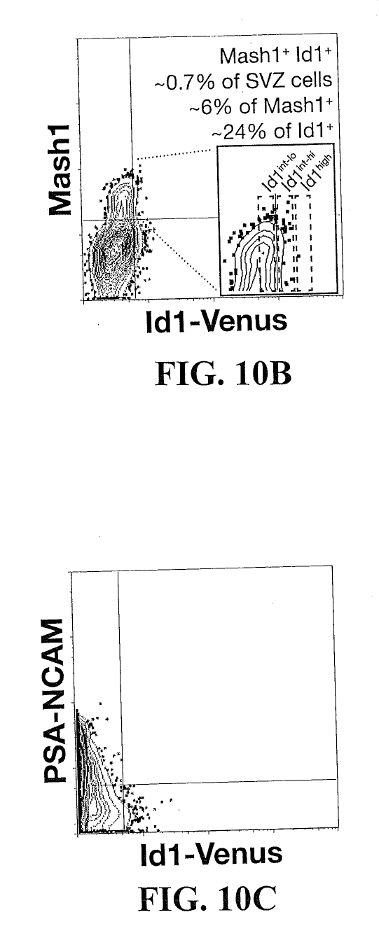

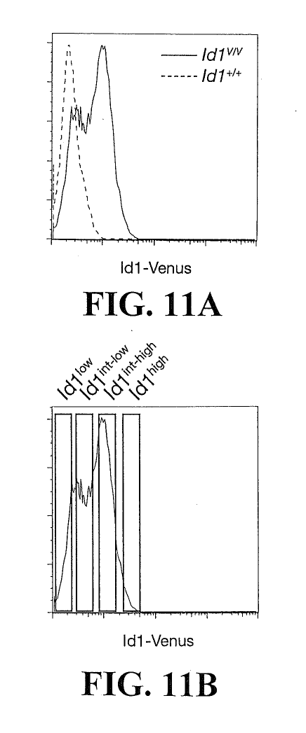

We further quantitated the Id1-expressing populations by flow cytometry. To that end, we used the Id1-VenusYFP fusion protein knock-in allele as described above (FIG. 1). The Id1-Venus fusion protein was functional in in vitro luciferase reporter assays, and the Id1 vallele could substitute for the Id1 wildtype allele in a genetic analysis (data not shown). Importantly, expression domains of the Id1-Venus fusion protein and the wildtype Id1 protein in Id1_{v/+} heterozygous mice and embryos overlapped without any significant difference, indicating that the Id1-Venus fusion protein faithfully recapitulates an Id1 expression pattern. For simplicity, we refer to the Id1-Venus expressing and non-expressing cells as Id1+ and Id1− cells, with finer classification into Id1_{intermediate-low}, Id1_{intermediate-high}, and Id1_{high} cells (FIG. 10B and FIGS. 11A and B).

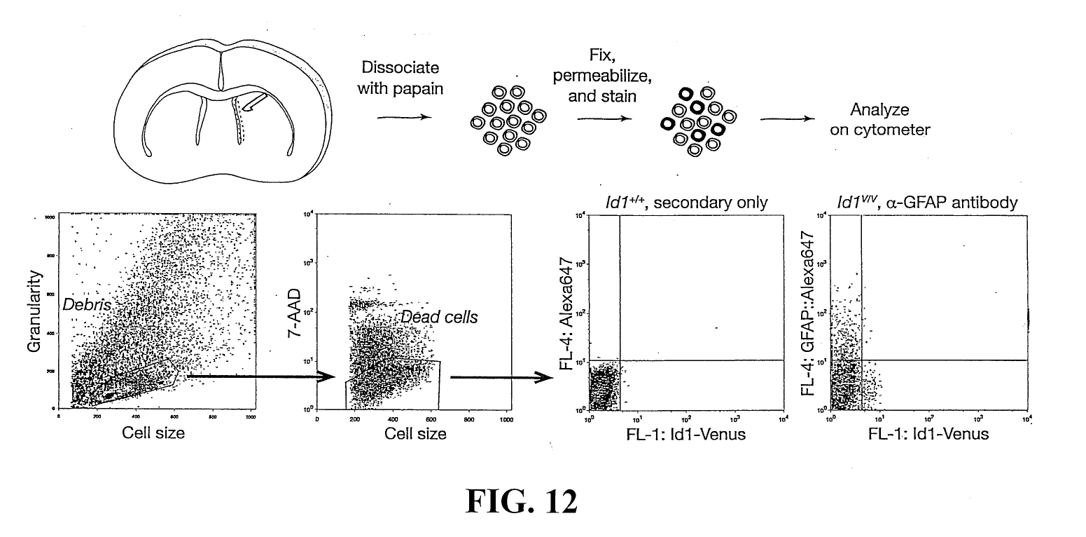

Subventricular tissue from Id1_{V/V} homozygous mice was microdissected from ˜1 mm coronal slices with a steel brain blocker (˜0.7 mm to ˜0.3 mm relative to Bregma), dissociated to single cells with papain, and analyzed by flow cytometry (FIG. 12). Because of the small tissue mass, ten minute incubation at 37° C. was sufficient to dissociate the tissue to single cells and preserved surface antigens from proteolysis. Centrifugation through a BSA or Percoll gradient removed some but not all debris. However, this also reduced cell yield and was unnecessary for phenotyping. Dissociated cells were resuspended in 2% BSA in PBS (wash buffer) and incubated for 5 min on ice. For intracellular staining, cells were fixed and permeabilized in fix/perm buffer (Cytofix/perm, BD) for 20 min on ice. Cells were blocked with normal goat IgG (Caltag) for 10 min on ice. Approximately 1 μg of antibodies per one million cells were added to the cells in the wash buffer with normal goat IgG and incubated for 30 min on ice. After washes, secondary antibodies (BD, Invitrogen) were incubated with the cells for 15 min on ice. After washes, cells were resuspended in HBSS with propidium iodide (2 μg/ml, Invitrogen) to discriminate dead/dying cells. For live/dead discrimination in fixed and permeabilized cells, a combination of 7-AAD and actinomycin D (Calbiochem) were used. In these stainings, dead cells were labeled with 7-AAD (1 μg/ml) prior to fixation and permeabilization, and actinomycin D was added to all buffers used subsequent to 7-AAD staining (1 μg/ml). Stained cells were analyzed on FACSCalibur, Aria, or MoFlo cytometers (BD, DakoCytomation) following standard practices. A sample gating is shown in FIG. 12 from the GFAP Id1− Venus analysis.

Flow cytometric quantitation of at least 20,000 cells indicated 3.1±0.64% of the cells of the microdissected tissue expressed Id1 (mean±standard deviation, n=14, 3 mice per n). Of these Id1-expressing cells, 16% were GFAP+ (FIG. 10A). Thus, the Id1-expressing subventricular astrocytes constituted only 0.49% of the cells of the microdissected subventricular tissue, a number close to 0.4% of subventricular cells estimated to be stem cells. Of the Id1-expressing cells, 24% were Mash1+ (FIG. 10B), though the majority of Mash1+ cells did not express Id1. Consistent with the immunohistological analyses, only one Id1_{high} cell in 20,000 subventricular cells dissociated from three mice expressed Mash1.

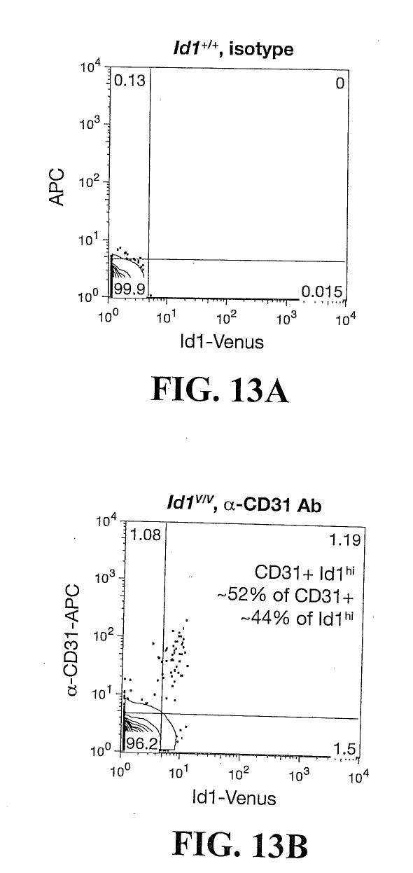

Moreover, most Id1_{intermediate-high} cells expressed lower levels of Mash1 than Id1_{intermediatelow} cells. Finally, PSA-NCAM+ cells did not express Id1 (FIG. 10C), consistent with the immunohistochemical analyses. In sum, of all subventricular Id1-expressing cells, 16% were GFAP+ astrocytes, 24% were Mash1+ cells, 10% were Olig2+ cells, and 44% were CD31+ endothelial cells (FIGS. 13A and B), with 6% unclassified.

Relative Quiescence of the Subventricular Id1_{high} Astrocytes

A one-hour pulse of the thymidine analog EdU indicated that the majority of the S-phase cells are Id1− by immunofluorescence (Id1− EdU+/all EdU+=95%). Further analyses with Ki67− which labels cells in G1−, S−, G2−, and M-phase-indicated that, consistent with the EdU labeling, the majority of the cycling Ki67+ were Id1− (Id1− Ki67+/all Ki67+=87%). Finally, analyses with Mcm2—which specifically labels cells in G1-phase and labels proliferating as well as relatively quiescent cells (Maslov et al., 2004)—indicated that the majority of the Mcm2+ cells were Id1− (Id1− Mcm2+/Mcm2+=87%). Of the Id1+ cells, 32% were EdU+. Id1_{high} cells were rarely EdU+ as evidenced by the observation that genetically labeled Id1_{high} cells were EdU−. The 32% of the Id1+ cells that are EdU+, therefore likely corresponded to the cycling Id1 intermediate Mash1+ or Olig2+ C type transit amplifying cells. A much greater percentage of the Id1_{high} cells were Ki67+/Mcm2+ than EdU+, suggesting that the some Id1_{high} cells may not be synthesizing DNA at a given moment in time but perhaps “paused” in other phases of the cell cycle, including G1. Consistent with the relative quiescence of these Id1_{high} cells, a seven-day infusion of EdU by miniosmotic pump labeled a greater percentage of Id1+ cells than a one-hour pulse.

The relative quiescence of the GFAP+ Id1_{high} cells was directly tested by six-day infusion of Ara-C, which ablates rapidly dividing C and A cells but spares the relatively quiescent B cells and quiescent non-stem-cell astrocytes (Doetsch et al., 1999a). The GFAP+ Id1_{high} astrocytes persisted after Ara-C ablation, consistent with their relative quiescence. However, the number of the round Id1+ nuclei was reduced by 83%. This is roughly consistent with the flow cytometric quantitation in which, of the Id1-expressing neural cells, 32% were GFAP+ Id1_{high} cells, and the remaining 68% were Mash1+ or Olig2+ Id1ᵢₙₜ transit amplifying cells. At twelve and forty-eight hours after Ara-C infusion, EdU was injected to label B cells activated to enter cell cycle. No EdU+ cells were observed immediately after Ara-C infusion. At twelve and forty-eight hours after Ara-C infusion, the number of EdU+ cells in the SVZ was greatly reduced relative to the non-AraC-infused mice. At these time points, the majority of the rare EdU+ cells were Id1+ (Id1+ EdU+/all EdU+=63%), although not all Id1+ cells were EdU+ (Id1+ EdU+/all Id1+=37%).

Subventricular GFAP+ Id1_{high} Astrocytes are B1 Type Cells

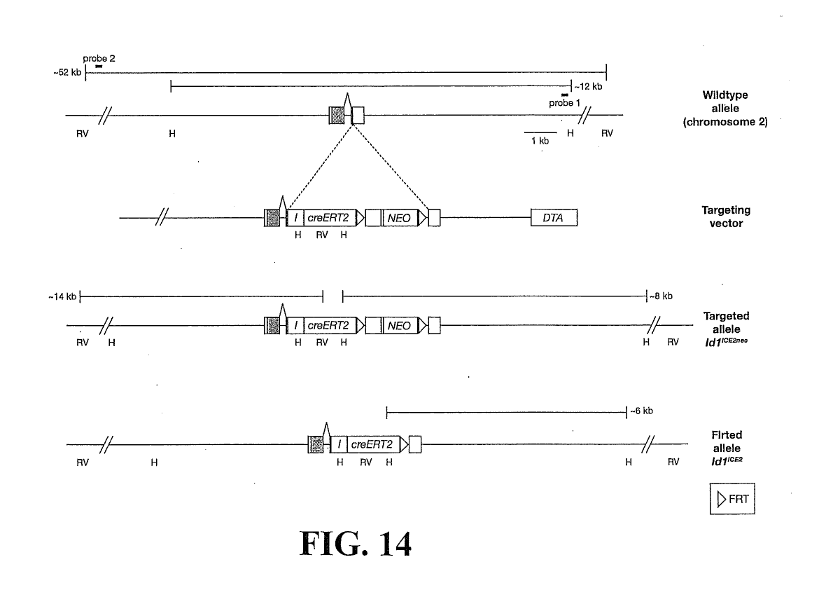

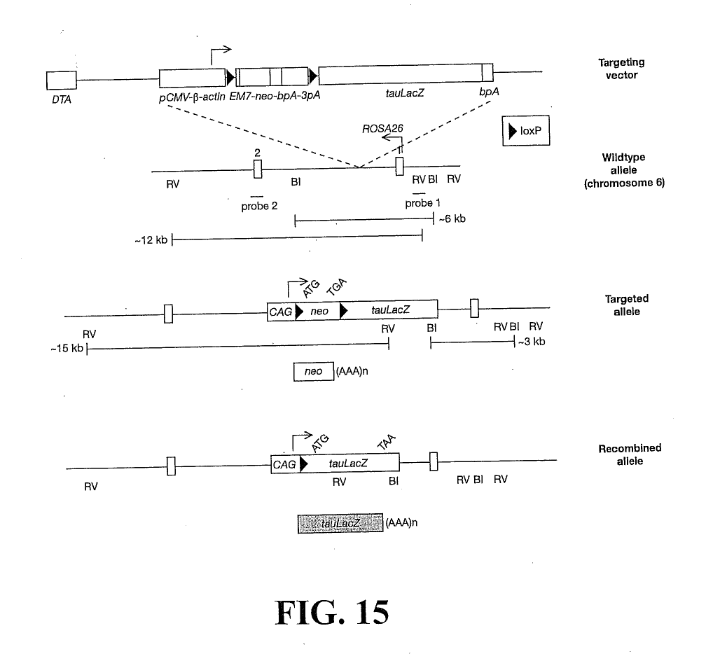

That high Id1 expression defined a relatively quiescent population of subventricular GFAP+ astrocytes capable of entering cell cycle raised the possibility that these GFAP+ Id1_{high} astrocytes may be the B1 type stem cells. Thus, the Id1-expressing cells were fate-mapped using the Id₁_{IRES-creERT2} and various reporter alleles (FIGS. 14, 15 and 16). In mice heterozygous for the Id1_{IRES-creERT2} allele, Id1-expressing cells express the tamoxifen-inducible cre recombinase creERT2 (Feil et al., 1997) from a bicistronic messenger RNA by means of an encephalomyocarditis virus internal ribosome entry site (EMCV IRES, (Kim et al., 1992)). The Id1 coding sequence was preserved in order to minimize interfering with Id1 expression levels, as Id1 haploinsufficiency could affect self-renewal and differentiation. Previous studies of the mouse olfactory system with gene-targeted bicistronic Olfactory Receptor-IRES-tauLacZ alleles have demonstrated that the EMCV IRES enables expression of a second cistron, albeit at a lower level, without altering the expression pattern of the targeted locus (for example, (Mombaerts et al,, 1996)). Because of the low creERT2 expression level, cre-mediated recombination most likely occurs in a small number of cells expressing comparatively higher levels of Id1 (see below and Discussion). In StLa mice, the CMV enhancer-chicken β-globin hybrid promoter (Niwa et al., 1991) drives the tauLacZ cre reporter allele.

Three days after the last tamoxifen dose, X-gal histochemistry of Id₁_{IRES-creERT2/+};StLa mouse sections revealed rare single cells with astrocytic morphology in the SVZ (n=3). In total, we examined 144 12-micron cryo-sections covering the anterior SVZ (from ˜1.8 mm to ˜−0.9 mm relative to Bregma), and found 10 single tau-β-gal+ subventricular astrocytic cells. A comparison of the number of X-gal+ cells to Id1-immunoreactive cells indicated <1% labeling efficiency in the brain, raising the possibility that recombination occurred only Id1_{high} Mash1− cells (see below and Discussion). These rare tau-β-gal+ cells in the SVZ were GFAP+ astrocytes. As expected, no X-gal+ cells with neuronal morphology nor tau-β-gal+ NeuN+ neurons were found in the OB at this time.

In the SVZ, the B1 type stem cells are organized in a “pinwheel” architecture and extend a long single basal process within the SVZ (Mirzadeh et al., 2008). Consistent with a B1 type identity of the genetically labeled cells, immunofluorescence of SVZ whole mounts from the Id1_{IRES-creERT2/+}; R26_{LSL-YFP} mice three days post-tamoxifen revealed rare YFP+ GFAP+ cells with the characteristic long single GFAP+ basal process (n=7). Moreover, the YFP+ GFAP+ astrocytes were located at the center of the pinwheel. The YFP+ GFAP+ astrocytes extended basal processes at least 90 microns long. B2 type astrocytes were never observed. Finally, all YFP+ GFAP+ astrocytes analyzed were Mash1− consistent with the claim that genetically labeled cells in these mice are Id1_{high} cells (n=13 cells).

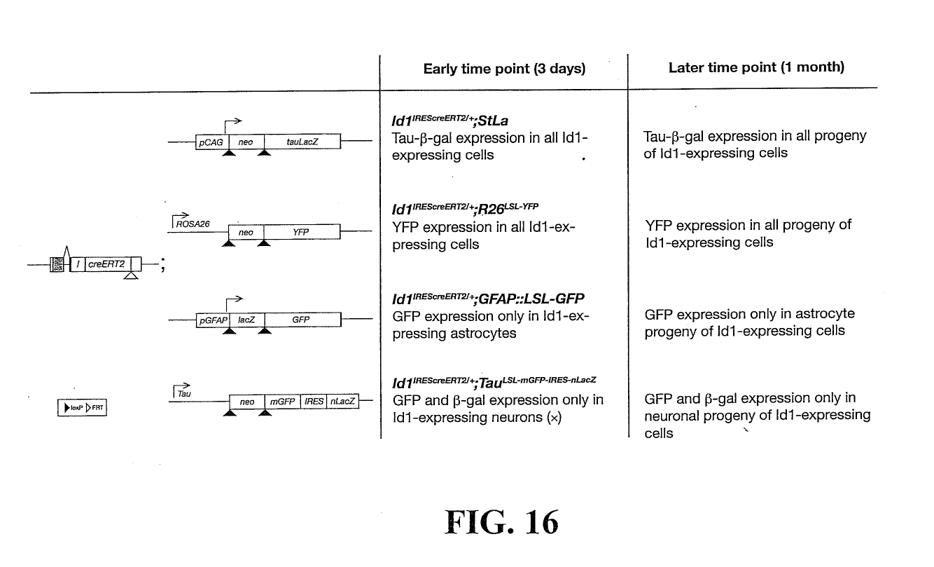

In addition to the anti-GFAP antibody, the GFAP::LSL-GFP transgenic mouse in which the ˜2.2 kb human GFAP promoter drives a GFP cre reporter ((Casper and McCarthy, 2006), FIG. 16) provided a genetic proof that the labeled cells are GFAP-expressing astrocytes. Immunofluorescence of SVZ whole mounts from the Id1_{IREScreERT2/+}; GFAP::LSL-GFP mice three days post-tamoxifen revealed rare GFP+ GFAP+ subventricular astrocytes (n=4). Consistent with a B1 type cell identity, this astrocyte extended a GFAP+ cell body to the center of the pinwheel and extended a single basal GFAP+ process to the nearby the vasculature. Moreover, the GFP+ GFAP+ astrocytes did not incorporate EdU during an one-hour pulse, indicating that these cells are relatively quiescent Id1_{high} B type cells rather than constitutively proliferating Id1ᵢₙₜ Mash1+/Dlx2+/Olig2+ C type cells.

Subventricular GFAP+ Id1_{high} B1 Type Astrocytes are Neurogenic Stem Cells

One and six months after the last tamoxifen dose, X-gal histochemistry of Id1_{IREScreERT2/+}; StLa and Id1_{IRES-creERT2/+};R26R mice sections revealed rare β-gal+ cells in the SVZ, RMS, and the OB. Six months after the last tamoxifen dose, in 72 12-micron sections examined, 4 single subventricular X-gal+ cells were found. Clusters of X-gal+ cells were never found, consistent with a cell division time longer than the time required for migration out of the SVZ, as was suggested by the cell cycle analysis. Some of the tau-β-gal+ cells in the RMS and OB of the Id1_{IRES-creERT2/+};StLa mice at one month post-tamoxifen were DCX+ neuroblasts with migratory morphology and NeuN+ neurons, respectively. Thus, under normal physiologic conditions during the month after tamoxifen administration, the subventricular GFAP+ Id1_{high} B1 type astrocytes gave rise to DCX+ neuroblasts that migrated to the OB and differentiated into NeuN+ neurons.

As some of the genetically labeled cells in the Id1_{IRES-creERT2/+};StLa mouse brains were endothelial cells, we utilized a neuron-specific reporter allele, Tau_{LSL-mGFP-IRES-nLacZ}, driven by the endogenous Tau (Mapt) promoter (Hippenmeyer et al., 2005) to specifically label genetically only the neuronal progeny of the Id1_{high} cells (FIG. 16). This enabled facile flow cytometric quantitation of the neuronal output at the steady-state from subventricular Id1_{high} B1 astrocytes to the olfactory bulbs by counting the number of GFP+ cells in dissociated olfactory bulbs.

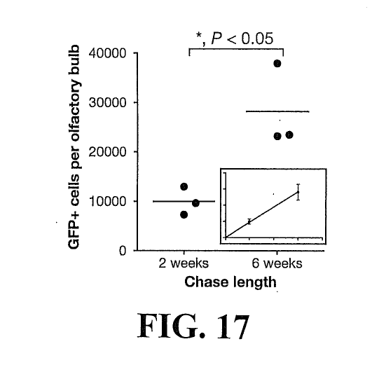

Flow cytometric quantitation at two weeks and six weeks post-tamoxifen indicated 9941±1636 GFP+ neurons per OB at two weeks (mean±SEM, n=3) and 28180±4866 neurons at six weeks (n=3), an approximately three-fold linear increase in the neuronal output (FIG. 17, P<0.05, unpaired two-tailed Student's t-test). Immunofluorescence of OB sections at one month post-tamoxifen confirmed the neuronal morphology of the GFP+ cells in the granular cell layer (FIG. 4E) and GABA expression.

Finally, we demonstrated with the Id1_{IRES-creERT2};Tau_{LSL-mGFP} mice that the subventricular Id1_{high} astrocytes are neurogenic stem cells in vivo. The compound heterozygous mice were gavaged with tamoxifen, then infused with Ara-C. This infusion ablates the rapidly dividing Id1ᵢₙₜ Mash1ᵢₙₜ C type cells as well as the A type neuroblasts, while the relatively quiescent Id1_{high} B type cells would persist. Twelve hours after Ara-C infusion, when some of the normally quiescent B type cells enter cell cycle, EdU was injected to label the B type cells in S-phase. Finally, after a two-week chase, analyses of the sections revealed GFP+ EdU+ neurons in the olfactory bulb. This experiment directly demonstrated that 1) the genetically-identified Id1_{high} B type cells are relatively quiescent and thus can persist through the antimitotic treatment, 2) these genetically-identified cells can enter cell cycle, and 3) these cells are undifferentiated and give rise to a neuron whose identity is reported by the Tau_{LSL-mGFP} knock-in allele.

Cultured Subventricular Id1_{high} Stem Cells Can Self-Renew Asymmetrically

Potentially, the GFAP+ Id1_{high} B1 type stem cell could self-renew asymmetrically and produce an asymmetric pair of progeny cells in vivo, i.e., a GFAP+ Id1_{high} B1 type stem cell and an Id1ᵢₙₜ Mash1ᵢₙₜ C type transit amplifying cell which differ subtly in the Id1 protein levels. Experiments indicated that the Id1 and Id1-Venus expression levels are heterogeneous in adherent adult neural stem/progenitor cell cultures (Glaser et al., 2007; Pollard et al., 2006) and that the subventricular lineages and the expression level gradients of at least two markers, Id1− Venus and Olig2, were recapitulated in vitro. These adherent radial glia-like tripotent cells derived from adult SVZ neurospheres were ˜100% nestin+, and importantly, free of non-neural cell types, e.g., endothelial cells. Thus, the Id1-Venun mouse and the adherent culture system provided a unique experimental system to examine the self-renewal behavior of the unperturbed Id1_{high} stem cells.

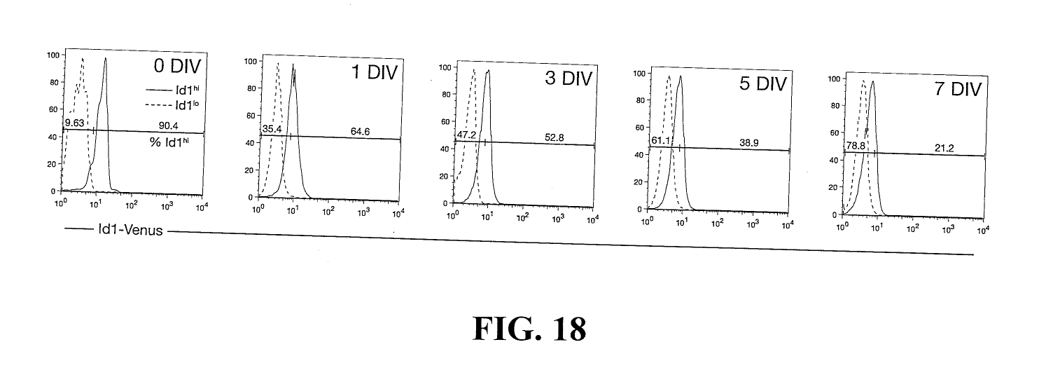

We examined the progeny of the Id1_{high} B-like stem and Id1_{low} A-like neuroblast cells (see FIGS. 11A and B) in adult adherent SVZ cell cultures by culturing the purified cells individually and measuring the percentage of Id1_{high}, Id1ᵢₙₜ, and Id1_{low} cells flow cytometrically. In serum-free medium supplemented with FGF-2 and EGF, the Id1_{high} stem cells generated Id1_{high} and Id1ᵢₙₜ cells (as evidenced by the leftward shift of the histogram concomitant with increase in cell density), but the Id1_{low} neuroblast cells generated only Id1_{low} cells and did not generate Id1_{high} or Id1ᵢₙₜ cells (FIG. 18).

Cell cycle parameters of the adherent Id1_{high} and Id1_{low} cells were measured by BrdU pulse-chase and flow cytometric quantitation. Consistent with the notion that Id1_{high} cells can self-renew asymmetrically, when the experimental data were modeled with population dynamics based on experimentally obtained cell cycle parameters of the Id1_{high} and Id1_{low} populations, the data closely resembled the population dynamics predicted of asymmetric self-renewal in at least 50% of the Id1_{high} cells (FIG. 8).

Consistent with an asymmetric self-renewal in which the Id1_{high} stem cells give rise to daughter cells with distinct Id1 levels, when Id1_{high} cells were FAC sorted and cultured at clonal density, Id1_{high} cells gave rise to clusters of cells expressing high and lower levels of Id1 (the latter undetectable by confocal microscopy).

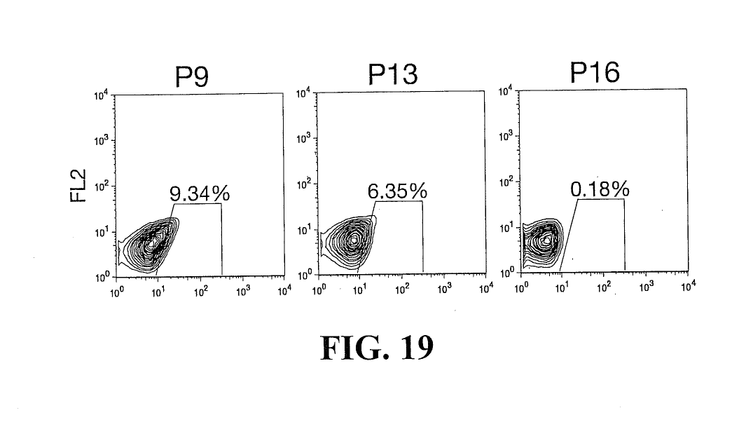

Finally, in further support of the conclusion that the Id1_{high} cells can self-renew asymmetrically at a population level, during serial passages the percentage of Id1_{high} cells were diluted out by the more rapidly proliferating Id1_{low} cells (FIG. 19), although obvious morphological changes to the predominantly bipolar radial glia-like morphology were absent even after forty passages, or four months, in culture.

Id Genes are Necessary for Self-Renewal, a Characteristic of Stem Cell Identity

The experiments above indicated that in addition to defining the B1 type astrocyte stem cells, high Id1 expression may be functionally important in the neural stem cell identity. We thus assayed the neurosphere-forming activity of the Id1_{high} and Id1_{low} fractions in early and later passage Id1_{V/V} adult adherent SVZ cell cultures. When Id1_{high} and Id1_{low} cells were FAC sorted and compared in clonal neurosphere assays, Id1_{high} cells generated more and larger spheres (FIG. 3A-C, sphere number, ˜2-fold increase, n=19, P<0.01; sphere size, ˜3.5-fold increase, n=19, P<0.0001; unpaired two-tailed student's t-test).

Upon passaging, only Id1_{high} cell-derived neurospheres formed secondary neurospheres (n=18). The Id1_{low} cell-derived spheres did not form secondary spheres. When cultured as adherent monolayer, Id1_{low} cells proliferated more rapidly than the Id1_{high} cells. Thus, only Id1_{high} cells were capable of self-renewing anchorage-independently.

The analyses above suggested high levels of Id1 are required for self-renewal in unperturbed cells, a characteristic of stem cell identity. However, adult Id1_{-/-} or Id3_{-/-} mice are viable and fertile, and neurosphere self-renewal is normal, suggesting redundancy or compensation. Thus, we asked whether both Id1 and Id3 are functionally required for neurosphere formation using the Id1-floxed conditional allele (FIG. 6). Neurospheres of Id1_{+/+};Id3_{+/+}, Id1_{fl/fl};Id³_{+/+}, and Id1_{fl/fl};Id3_{-/-} genotype were nucleofected with cre recombinase cDNA (˜50% efficiency by flow cytometry). Ablation of Id1 and Id3, but not Id1 alone, was sufficient to reduce secondary neurosphere formation by ˜50% (FIG. 6C), a number consistent with near complete absence of self-renewal in the transfected population. Genomic PCR detected the unrecombined floxed allele in neurospheres that formed.

Subgranular GFAP+ Id1_{high} Astrocytes are Also Neurogenic Stem Cells

To determine whether high Id1 expression is a general characteristic of the GFAP+ astrocyte stem cells, we examined Id1 expression and the progeny of Id1-expressing cells in the hippocampal dentate gyrus. Id1+ cells were evident in the subgranular layer, and these cells were GFAP+ (FIG. 7A), although a clear long apical GFAP+ process wasn't apparent for all Id1+ subgranular cells. These Id1+ cells also expressed nestin, a marker of neural stem/progenitor cells (FIG. 7B). Consistent with the Id1 gradient evident in the subventricular neurogenic lineages, subgranular Id1+ cells never expressed DCX and NeuN. Finally, in Id1_{IRES-creERT2/+};StLa mice one month post-tamoxifen, tau-β-gal+ NeuN+ cells with neuronal morphology were evident in the granuar cell layer.

The results above demonstrate that GFAP+ Id1_{high} cells are the bona fide B1 type stem cells of the adult murine subventricular neurogenic lineages. First, the GFAP+ Id1_{high} cells are rare. Previous estimation of the endogenous NSC number (Morshead et al., 1998)—although thought to be an overestimation (Golnohammadi et al., 2008)—suggested that merely 0.4% of the subventricular cells are stem cells, based on number of cells that entered the cell cycle after high-dose ₃Hthymidine kill. Flow cytometric quantitation of the GFAP+ Id1_{high} cells in the anterior SVZ indicated that these cells constitute merely 0.5% of the subventricular cells. Second, the GFAP+ Id1_{high} cells are capable of proliferation, though a majority of them are not in S-phase at the ages analyzed. This suggests that these cells are quiescent, dormant, or perhaps paused in cell cycle. Indeed, the GFAP+ Id1_{high} cells persisted after Ara-C infusion, consistent with relative quiescence. However, the exact cell cycle status of these cells remain unclear. A majority of the S-phase cells as identified by a short pulse of the thymidine analog were Id1−. However, a greater percentage of the Id1− expressing cells expressed Ki67 or Mcm2 which label a larger population of cycling cells than a one-hour pulse of thymidine analog. Thus, although the Id1_{high} cells may be capable of proliferation, as evidenced by EdU incorporation in a very low percentage of cells and Ki67 or Mcm2 expression, they may be paused in phases other than S-phase, including G1. This result is consistent with the recent finding that the ventricle-contacting B1 type cells are found in all phases of the cell cycle (Mirzadeh et al., 2008). Third, the GFAP+ Id1_{high} cells generate differentiated neuronal and glial progeny (in vitro), from which we infer that the cells are undifferentiated and multipotent. Fourth, the neuronal output from the Id1_{high} cells increases linearly from two weeks to six weeks post-tamoxifen. Previous studies indicate that when rapidly dividing cells are labeled by ₃H-thymidine once per day for four days, neuronal output peaks at ˜15 days then drops until ˜45 days (Petreanu and Alvarez-Buylla, 2002). In contrast, when relatively quiescent stem cells are labeled by stereotaxic lentiviral injection and measured by bioluminescence imaging, neuronal output continues to increase 2-6 weeks post-labeling, with a linear increase between 4-6 weeks (Reumers et al., 2008). Thus, the linear increase in neuronal output measured in the Id1_{IREScreERT2}; Tau_{LSL-mGFP} is consistent with a neural stem cell identity of the Id1_{high} cells as opposed to a transit amplifying cell identity. Fifth, the SVZ GFAP+ Id1_{high} cells maintain themselves for at least six months after labeling.

Taken together, these findings indicate that the subventricular GFAP+ astrocytes expressing high levels of Id1 are the long-sought-after B1 type neural stem cells.

The Distinct Characteristics of Id1_{high}, Id1_{intermediate}, and Id1_{low} Cells

We propose a model in which the cells of the subventricular neurogenic lineages express a gradually decreasing level of Id1. That is, subventricular neurogenesis proceeds from GFAP+ Id₁_{high} B1 type stem cells to Id1_{int/low} Mash1_{int/high} C type transit amplifying cells to GFAP− Id₁_{low} Mash₁_{low} Pax6ₕᵢ PSA-NCAM/DCX+ A type neuroblast cells. This gradient was evident in vivo and in vitro. Fortuitously, the low frequency partial fate mapping of the subventricular Id1-expressing cells enabled genetic identification and fate mapping of the cells expressing comparatively higher levels of Id1, for the following reason. The probability of creERT2-mediated recombination depends upon its expression level (Kuhbandner et al., 2000). Even in Id1_{high} cells, Id1 is expressed at a level lower than other gene products, e.g., GFAP (H. Nam, unpublished work), and the creER protein level is further lowered by translation initiated from the EMCV IRES (Bochkov and Palmenberg, 2006). As creER-mediated recombination activity depends upon its expression level, consequently, in cells expressing comparatively high levels of Id1, i.e., the Id1_{high} cells, is creER-mediated activation of the reporter gene more likely to occur.

Moreover, as the labeling efficiency is very low (˜1% of Id1-immunoreactive cells), even if the Id1_{low} cells were labeled, the majority of the genetically labeled cells are more likely to be Id1_{high} cells than Id1ᵢₙₜ cells. Indeed, the GFP+ cells in Id1_{IREScreERT2/+}; GFAP::LSL-GFP mice were Mash1− (data not shown), consistent with the immunofluorescence analyses in which the Id1_{high} cells were Mash1−.

Furthermore, in vitro experiments directly demonstrated the Id1 gradient in unperturbed cells of subventricular neurogenic lineages. In vitro, the Id1_{high} B-like stem cells produced Id1_{high} B-like stem and more differentiated Id1ᵢₙₜ C-like transit amplifying progeny cells, which in turn produced Id1_{low} A-like neuroblast cells. Consistent with this, no difference in C cell markers, Dlx2, Mash1, or Olig2, was evident between the B-like Id1_{high} and A-like Id1_{low} populations. According to the model, the Id1ᵢₙₜ cells corresponds to the C type transit amplifying cells. Indeed, when Id1_{high}, Id1ᵢₙₜ, and Id1_{low} cells were FAC sorted and analyzed with sub-saturating amount of anti-Olig2 antibody, a C cell marker, the percentage of Olig2_{high} cells was highest in the Id1ᵢₙₜ fraction and lower in the Id1_{high} and Id1_{low} fractions. Thus, the Id1 gradient was directly demonstrated in the progression from cultured stem to differentiated precursor cells.

Importantly, these results suggest a threshold of Id1 levels for “stemness” That is, distinct characteristics evident in Id1_{high}, Id1ᵢₙₜ, and Id1_{low} cells are expressions of subtle differences in the Id1 levels. Subtle differences in the Id1 level could contribute to the likelihood of a progeny cell altering its fate in concert with stochastic mechanism of cell fate determination, perhaps by altering the activity or expression of basic-helix-loop-helix transcription factors such as Mash1 or Olig2.

The Roles and Contributions of Id Genes in Adult Stem Cell Identity

In the neurosphere self-renewal assay, ˜50% nucleofection of the cre recombinase cDNA elicited a ˜50% reduction in self-renewal, suggesting a cell-autonomous requirement for the Id genes in self-renewal. In a recent report, Id4 overexpression was sufficient to confer self-renewal capacity to postnatal astrocytes (Jeon et al., 2008). Thus, taken together, Id genes are necessary and sufficient for the self-renewal capacity in an anchorage-independent manner, a cardinal in vitro characteristic of stem cell identity. However, Id1 is dispensable for proliferation. Despite the lack of self-renewal capacity evident in the Id1_{Δ/Δ};Id3_{-/-} cells, these cells were quite capable of proliferation in an anchorage-dependent manner. Indeed, Id1_{low} cells proliferated more rapidly than Id1_{high} cells in adherent cultures consistent with the interpretation that Id1 is dispensable for proliferation. Id proteins counter positive-acting basic-helix-loop-helix proteins in vitro and in vivo (reviewed in (Perk et al., 2005)). Sequestering these factors from transcriptionally productive interactions with the chromatin and thus preventing subsequent commitment to a given cell fate may be one central role for Id proteins in maintaining stem cell identity. One important direct downstream target of Id1 is p16, a gene implicated in modulation of age-dependent decrease in self-renewal of neural and hematopoietic stem cells (Janzen et al., 2006; Molofsky et al., 2006). In Id1_{-/-},Id3_{-/-} mice, an increase in p16 is observed in the brain consequent to or concomitant with precocious differentiation (Lyden et al., 1999). Conversely, Id1 overexpression in vitro is sufficient to reduce p16 (Ohtani et al., 2001; Ouyang et al., 2002).

However, the roles and contributions of each Id gene product to the stem cell identity are likely to be complex. The findings that in unperturbed Id1_{low} cells, levels of another Id family member, Id3—often hypothesized to compensate for Id1 deletion—were similar to that observed in Id1_{high} cells but were insufficient to confer self-renewal capacity to these cells suggest that Id1_{low} cells lack other components of the self-renewal mechanism and that Id3 alone cannot not compensate for the missing components.

In summary, our results indicate that high levels of Id1 expression defines the B1 type neural stem cell identity. Ongoing work shows that stem cells of another neural stem cell niche, e.g., olfactory epithelium, and a non-neural stem cell niche, e.g., intestinal crypt, express high levels of Id1, showing a broad and conserved role of Id1_{high} stem cells and Id genes within and outside the central nervous system. Elucidating further the roles, contributions, and behavior of the Id gene products may ultimately shed light on the intricate molecular cell biological process of adult stem cell self-renewal during normal and pathologic homeostasis.

Method for Identifying Cells

In accordance with one aspect of there is provided a method for identifying cells as potential stem cells comprising the step of screening the cells for expression of Id1. This method can be practiced using any known method for detection of Id1 expression, including without limitation those discussed above, and those disclosed in U.S. Pat. No. 7,247,719, US Patent Publications 20030022871 and 20040014114, and Lin, C. Q., Singh, J., Murata, K., Itahana, Y., Parrinello, S., Liang, S. H., Gillett, C. E., Campisi, J. & Desprez, P. Y. (2000). Cancer Res, 60, 1332-40; Singh, J., Murata, K., Itahana, Y. & Desprez, P. Y. (2002). Oncogene, 21, 1812-22; and Schoppmann, S. F., Schindl, M., Bayer, G., Aumayr, K., Dienes, J., Horvat, R., Rudas, M., Gnant, M., Jakesz, R. & Bimer, P. (2003). Int J Cancer, 104, 677-82.

Thus, detection of Id1 expression can be carried out using Id1 specific amplification primers, Id1 specific polynucleotide hybridization probes, Id1 binding antibodies, and Id-1 binding molecules such as tetracycline, using assay techniques such as Northern, Southern and Western Blots, and immunohistochemical analysis.

In the case of B1 type adult neural stem cells an additional screening step for GFAP may be employed in combination with the testing for Id1 expression. Other markers associated with other stem cell lineages may also be employed with Id1 screening.

Method For Isolating Cells

The cells identified as expressing Id1 from a sample of putative stem cells are suitably separated from cells that that do not, and thus provide a sample that has a higher level of possible stem cells than the original sample. These cells may then be further purified using known cell isolation techniques, including selection microarrays for cells expressing appropriate cell surface markers. See for example U.S. Pat. Nos. 7,320,872 and 6,852,533.

Applying the methods of U.S. Pat. No. 6,852,533, a mixture of cells from which stem cells are to be isolated is exposed to a molecule that binds specifically to the Id1 marker characteristic of stem cells. The binding molecule is preferably an antibody or a fragment of an antibody.

The cells that express the antigen marker bind to the binding molecule. The binding molecule distinguishes the bound cells from unbound cells, permitting separation and isolation. If the bound cells do not internalize the molecule, the molecule may be separated from the cell by methods known in the art. For example, antibodies may be separated from cells by a short exposure to a solution having a low pH, or with a protease such as chymotrypsin.

The molecule used for isolating the purified populations of stem cells is advantageously conjugated with labels that expedite identification and separation. Examples of such labels include magnetic beads; biotin, which may be identified or separated by means of its affinity to avidin or streptavidin; fluorochromes, which may be identified or separated by means of a fluorescence-activated cell sorter (FACS, see below), and the like.

In one embodiment, the binding molecule is attached to a solid support. Some suitable solid supports include nitrocellulose, agarose beads, polystyrene beads, hollow fiber membranes, magnetic beads, and plastic petri dishes. For example, the binding molecule can be covalently linked to Pharmacia Sepharose 6 MB macro beads. The exact conditions and duration of incubation for the solid phase-linked binding molecules with the crude cell mixture will depend upon several factors specific to the system employed, as is well known in the art. Cells that are bound to the binding molecule are removed from the cell suspension by physically separating the solid support from the remaining cell suspension. For example, the unbound cells may be eluted or washed away with physiologic buffer after allowing sufficient time for the solid support to bind the stem cells.

The bound cells are separated from the solid phase by any appropriate method, depending mainly upon the nature of the solid phase and the binding molecule. For example, bound cells can be eluted from a plastic petri dish by vigorous agitation. Alternatively, bound cells can be eluted by enzymatically “nicking” or digesting an enzyme-sensitive “spacer” sequence between the solid phase and an antibody. Suitable spacer sequences bound to agarose beads are commercially available from, for example, Pharmacia. The eluted, enriched fraction of cells may then be washed with a buffer by centrifugation and preserved in a viable state at low temperatures for later use according to conventional technology. The cells may also be used immediately, for example by being infused intravenously into a recipient.

In a particularly preferred variation of the method described above, blood is withdrawn directly from the circulating peripheral blood of a donor. The blood is percolated continuously through a column containing the solid phase-linked Id1 binding molecule to capture stem cells. The stem cell-depleted blood is returned immediately to the donor's circulatory system by methods known in the art, such as hemapheresis. The blood is processed in this way until a sufficient number of stem cells binds to the column. The stem cells are then isolated from the column by methods known in the art. This method allows rare peripheral blood stem) cells to be harvested from a very large volume of blood, sparing the donor the expense and pain of harvesting bone marrow and the associated risks of anesthesia, analgesia, blood transfusion, and infection. Other methods for isolating the purified populations of stem cells are also known. Such methods include magnetic separation with antibody-coated magnetic beads, and “panning” with an antibody attached to a solid matrix.

In an embodiment, a labeled Id1 binding molecule is bound to the stem cells, and the labeled cells are separated by a mechanical cell sorter that detects the presence of the label. The preferred mechanical cell sorter is a fluorescence activated cell sorter (FACS). FACS machines are commercially available. Generally, the following FACS protocol is suitable for this procedure.

In another embodiment, cells are isolated using a magnetic cell sorting technique of the type described in U.S. Pat. No. 7,302,872. Cells to be sorted in culture medium are combined with an antibody against Id1 and incubated. The cells are then washed and combined with magnetic beads conjugated to an antibody specific for the antibody against Id1 employed, and incubated. After incubation, the culture medium is placed in a magnetic separator for collection of cells.

In specific embodiments, of the invention, where B1 type adult neural stem cells are desired one or more additional isolation steps may be used in combination with isolation based on Id1 expression. The first type of isolation step uses GFAP as a positive marker of B1 type adult neural stem cells. A sample containing putative stem cells of this type is suitably enriched using a GFAP-specific binding molecule as a positive selection tool. Furthermore, as noted above, PSA-NCAM+ neuroblasts and NeuN+ neurons were never seen to express Id1. Thus, a negative selection can be performed to enrich samples of putative stem cells prior to the actual isolation step by selecting out and removing cells that are for example by passing the sample through a solid support with binding molecules for these markers.

Isolated/Enriched Cells

The result of the isolation process discussed above is a mixture of cells, in which adult stem cells that express Id1 are enriched relative to a starting tissue extract. Thus, a further aspect of the present invention is a cell preparation in which the percentage of cells expressing Id1 are enriched at least 2 fold, preferably 5-fold, 10-fold or more, relative to a starting cell preparation obtained from an organism. A further aspect of the invention is a cell preparation in which the percentage of cells expressing both Id1 and GFAP are enriched at least 2 fold, preferably 5-fold, 10-fold or more, relative to a starting cell preparation obtained from an organism.

In one embodiments, the present invention provides a cell preparation containing B1 type adult neuronal stem cells, for example human cells, enriched at least 2 fold, preferably 5-fold, 10-fold or more, relative to a starting cell preparation obtained from the organism. In a specific embodiment, the at least 1%, and more preferably at least 2% of the cells in the cell preparation are B1 type adult neuronal stem cells.

Identification of Promoters or Inhibitors of Differentiation

Once a sample with purified stem cells is obtained, these cells may be cultured and used in in vitro testing of compounds for function as promoters or inhibitors of differentiation. By way of non-limiting example, US Patent Publication No. 20080057530 discloses a methodology for discovering neurogenic drugs in which test agents are exposed differentiating neural progenitor cells and measuring the effect of the agent on overall cell number and./or the number of neurons. Cells isolated using the method of the present invention from neural tissue can be used in this assay. See for example, U.S. Pat. Nos. 7,101,709, 7,070,996, and 6,497,872,

Studies on Stem-Ness

As noted above, there are some 1300 genes differentially expressed in the Id1 vs non-Id1 cells using microarrays. Ultimately, some of these genes could be used to manipulate stem cells by maintaining “stemness” in culture, or that would create stem cells from differentiated cells, or shut down “stemness” in cancer stem cells.

Screening of these genes is a two step process. Where the function of the gene is not known, an shRNA screen, knocking out each one individually to see the functional effect can be used. Then, for gene(s) that are required to reach a certain functional end, a small molecule high throughput screen is conducted to identify therapeutic agents that promote or inhibit differentiation.

Therapeutic Uses

Therapeutic uses for stem cells are disclosed in numerous places in the scientific and patent literature. By way of non-limiting example, stem cells identified using the methods of this invention and subsequently cultured can be used as described in US Patent Publication 20080090765, and U.S. Pat. Nos. 7,320,872, 7,150,990, 6,497,872, and 6,866,843

All of the patents and publications referred to herein are incorporated herein by reference in their entirety.

### References

Ahn, S., and Joyner, A. L. (2005). In vivo analysis of quiescent adult neural stem cells responding to Sonic hedgehog. Nature 437, 894-897.

Altman, J. (1969). Autoradiographic and histological studies of postnatal neurogenesis. IV. Cell proliferation and migration in the anterior forebrain, with special reference to persisting neurogenesis in the olfactory bulb. J Comp Neurol 137, 433-457.

Altman, J., and Das, G. D. (1965). Autoradiographic and histological evidence of postnatal hippocampal neurogenesis in rats. J Comp Neurol 124, 319-335.

Benezra, R., Davis, R. L., Lockshon, D., Turner, D. L., and Weintraub, H. (1990). The protein Id: a negative regulator of helix-loop-helix DNA binding proteins. Cell 61, 49-59.

Bochkov, Y. A., and Palmenberg, A. C. (2006). Translational efficiency of EMCV IRES in bicistronic vectors is dependent upon IRES sequence and gene location. Biotechniques 41, 283-284, 286, 288 passim.

Capela, A., and Temple, S. (2002). LeX/ssea-1 is expressed by adult mouse CNS stem cells, identifying them as nonependymal. Neuron 35, 865-875.

Carleton, A., Petreanu, L. T., Lansford, R., Alvarez-Buylla, A., and Lledo, P. M. (2003). Becoming a new neuron in the adult olfactory bulb. Nat Neurosci 6, 507-518.

Casper, K. B., and McCarthy, K. D. (2006). GFAP-positive progenitor cells produce neurons and oligodendrocytes throughout the CNS. Mol Cell Neurosci 31, 676-684.

Chiasson, B. J., Tropepe, V., Morshead, C. M., and van der Kooy, D. (1999). Adult mammalian forebrain ependymal and subependymal cells demonstrate proliferative potential, but only subependymal cells have neural stem cell characteristics. J Neurosci 19, 4462-4471.

Coslun, V., Wu, H., Blanchi, B., Tsao, S., Kim, K., Zhao, J., Biancotti, J. C., Hutnick, L., Krueger, R. C., Jr., Fan, G., et al. (2008). CD133+ neural stem cells in the ependyma of mammalian postnatal forebrain. Proc Natl Acad Sci U S A 105, 1026-1031.

F. Doetsch, J. M. Garcia-Verdugo, A. Alvarez-Buylla (1997), J Neurosci 17, 5046.

Doetsch, F., Caille, I., Lim, D. A., Garcia-Verdugo, J. M., and Alvarez-Buylla, A. (1999a). Subventricular zone astrocytes are neural stem cells in the adult mammalian brain. Cell 97, 703-716.

Doetsch, F., Garcia-Verdugo, J. M., and Alvarez-Buylla, A. (1999b). Regeneration of a germinal layer in the adult mammalian brain. Proc Natl Acad Sci U S A 96, 11619-11624.

Doetsch, F., Petreanu, L., Caille, I., Garcia-Verdugo, J. M., and Alvarez-Buylla, A. (2002). EGF converts transit-amplifying neurogenic precursors in the adult brain into multipotent stem cells. Neuron 36, 1021-1034.

Eriksson, P. S., Perfilieva, E., Bjork-Eriksson, T., Alborn, A. M., Nordborg, C., Peterson, D. A., and Gage, F. H. (1998). Neurogenesis in the adult human hippocampus. Nat Med 4, 1313-1317.

Feil, R., Wagner, J., Metzger, D., and Chambon, P. (1997). Regulation of Cre recombinase activity by mutated estrogen receptor ligand-binding domains. Biochemical and biophysical research communications 237, 752-757.

Fraidenraich, D. et al.(2004) Science 306, 247.

Garcia, A. D., Doan, N. B., Imura, T., Bush, T. G., and Sofroniew, M. V. (2004). GFAP-expressing progenitors are the principal source of constitutive neurogenesis in adult mouse forebrain. Nat Neurosci 7, 1233-1241.

Glaser, T., Pollard, S. M., Smith, A., and Brustle, O. (2007). Tripotential differentiation of adherently expandable neural stem (NS) cells. PLoS ONE 2, e298.

Goldman, S. A., and Nottebohm, F. (1983). Neuronal production, migration, and differentiation in a vocal control nucleus of the adult female canary brain. Proc Natl Acad Sci U S A 80, 2390-2394.

Golmohammadi, M. G., Blackmore, D. G., Large, B., Azari, H., Esfandiary, E., Paxinos, G., Franklin, K. B., Reynolds, B. A., and Rietze, R. L. (2008). Comparative analysis of the frequency and distribution of stem and progenitor cells in the adult mouse brain. Stem cells (Dayton, Ohio) 26, 979-987.

Hippenmeyer, S., Vrieseling, E., Sigrist, M., Portmann, T., Laengle, C., Ladle, D. R., and Arber, S. (2005). A developmental switch in the response of DRG neurons to ETS transcription factor signaling. PLoS biology 3, e159.

Ihrie, R. A., and Alvarez-Buylla, A. (2008). Cells in the astroglial lineage are neural stem cells. Cell and tissue research 331, 179-191.

V. Jankovic, A. Ciarrocchi, P. Boccuni et al., Proc Natl Acad Sci U S A 104 (4), 1260 (2007).

Janzen, V., Forkert, R., Fleming, H. E., Saito, Y., Waring, M. T., Dombkowski, D. M., Cheng, T., DePinho, R. A., Sharpless, N. E., and Scadden, D. T. (2006). Stem-cell ageing modified by the cyclin-dependent kinase inhibitor p 16INK4a. Nature 443, 421-426.

Jeon, H. M., Jin, X., Lee, J. S., Oh, S. Y., Sohn, Y. W., Park, H. J., Joo, K. M., Park, W. Y., Nam, D. H., DePinho, R. A., et al. (2008). Inhibitor of differentiation 4 drives brain tumor-initiating cell genesis through cyclin E and notch signaling. Genes Dev 22, 2028-2033.

Johansson, C. B., Momma, S., Clarke, D. L., Risling, M., Lendahl, U., and Frisen, J. (1999). Identification of a neural stem cell in the adult mammalian central nervous system. Cell 96, 25-34.

Kaplan, M. S., McNelly, N. A., and Hinds, J. W. (1985). Population dynamics of adult-formed granule neurons of the rat olfactory bulb. J Comp Neurol 239, 117-125.

Kim, D. G., Kang, H. M., Jang, S. K., and Shin, H. S. (1992). Construction of a bifunctional mRNA in the mouse by using the internal ribosomal entry site of the encephalomyocarditis virus. Mol Cell Biol 12, 3636-3643.

Kornack, D. R., and Rakic, P. (2001). The generation, migration, and differentiation of olfactory neurons in the adult primate brain. Proc Natl Acad Sci U S A 98, 4752-4757.

Kuhbandner, S., Brummer, S., Metzger, D., Chambon, P., Hofmann, F., and Feil, R. (2000). Temporally controlled somatic mutagenesis in smooth muscle. Genesis 28, 15-22.

Kuo, C. T., Mirzadeh, Z., Soriano-Navarro, M., Rasin, M., Wang, D., Shen, J., Sestan, N., Garcia-Verdugo, J., Alvarez-Buylla, A., Jan, L. Y., et al. (2006). Postnatal deletion of Numb/Numblike reveals repair and remodeling capacity in the subventricular neurogenic niche. Cell 127, 1253-1264.

Laywell, E. D., Rakic, P., Kukekov, V. G., Holland, E. C., and Steindler, D. A. (2000). Identification of a multipotent astrocytic stem cell in the immature and adult mouse brain. Proc Natl Acad Sci U S A 97, 13883-13888.

P. Liu, N. A. Jenkins, and N. G. Copeland, Genome Res 13 (3), 476 (2003).

Lois, C., and Alvarez-Buylla, A. (1994). Long-distance neuronal migration in the adult mammalian brain. Science 264, 1145-1148.

Luskin, M. B. (1993). Restricted proliferation and migration of postnatally generated neurons derived from the forebrain subventricular zone. Neuron 11, 173-189.

Lyden, D., Young, A. Z., Zagzag, D., Yan, W., Gerald, W., O'Reilly, R., Bader, B. L., Hynes, R. O., Zhuang, Y., Manova, K., et al. (1999). Id1 and Id3 are required for neurogenesis, angiogenesis and vascularization of tumour xenografts. Nature 401, 670-677.

Maslov, A. Y., Barone, T. A., Plunkett, R. J., and Pruitt, S. C. (2004). Neural stem cell detection, characterization, and age-related changes in the subventricular zone of mice. J Neurosci 24, 1726-1733.

Merkle, F. T., Mirzadeh, Z., and Alvarez-Buylla, A. (2007). Mosaic organization of neural stem cells in the adult brain. Science 317, 381-384.

Mirzadeh, Z., Merkle, F. T., Soriano-Navarro, M., Garcia-Verdugo, J. M., and Alvarez-Buylla, A. (2008). Neural stem cells confer unique pinwheel architecture to the ventricular surface in neurogenic regions of the adult brain. Cell Stem Cell 3, 265-278.

Molofsky, A. V., Slutsky, S. G., Joseph, N. M., He, S., Pardal, R., Krishnamurthy, J., Sharpless, N. E., and Morrison, S. J. (2006). Increasing p16INK4a expression decreases forebrain progenitors and neurogenesis during ageing. Nature 443, 448-452.

Mombaerts, P., Wang, F., Dulac, C., Chao, S. K., Nemes, A., Mendelsohn, M., Edmondson, J., and Axel, R. (1996). Visualizing an olfactory sensory map. Cell 87, 675-686.

Morshead, C. M., Craig, C. G., and van der Kooy, D. (1998). In vivo clonal analyses reveal the properties of endogenous neural stem cell proliferation in the adult mammalian forebrain. Development 125, 2251-2261.

Morshead, C. M., Reynolds, B. A., Craig, C. G., McBurney, M. W., Staines, W. A., Morassutti, D., Weiss, S., and van der Kooy, D. (1994). Neural stem cells in the adult mammalian forebrain: a relatively quiescent subpopulation of subependymal cells. Neuron 13, 1071-1082.

Nacher, J., Crespo, C., and McEwen, B. S. (2001). Doublecortin expression in the adult rat telencephalon. Eur J Neurosci 14, 629-644.

Nagai, T., Ibata, K., Park, E. S., Kubota, M., Mikoshiba, K., and Miyawaki, A. (2002). A variant of yellow fluorescent protein with fast and efficient maturation for cell biological applications. Nat Biotechnol 20, 87-90.

Niwa, H., Yamamura, K., and Miyazaki, J. (1991). Efficient selection for high-expression transfectants with a novel eukaryotic vector. Gene 108, 193-199.

Ohtani, N., Zebedee, Z., Huot, T. J., Stinson, J. A., Sugimoto, M., Ohashi, Y., Sharrocks, A. D., Peters, G., and Hara, E. (2001). Opposing effects of Ets and Id proteins on p16INK4a expression during cellular senescence. Nature 409, 1067-1070.

Ouyang, X. S., Wang, X., Ling, M. T., Wong, H. L., Tsao, S. W., and Wong, Y. C. (2002). Id-1 stimulates serum independent prostate cancer cell proliferation through inactivation of p16(INK4a)/pRB pathway. Carcinogenesis 23, 721-725.

Paton, J. A., and Nottebohm, F. N. (1984). Neurons generated in the adult brain are recruited into functional circuits. Science 225, 1046-1048.

Perk, J., Gil-Bazo, I., Chin, Y., de Candia, P., Chen, J. J., Zhao, Y., Chao, S., Cheong, W., Ke, Y., Al-Ahmadie, H., et al. (2006). Reassessment of id1 protein expression in human mammary, prostate, and bladder cancers using a monospecific rabbit monoclonal anti-idl antibody. Cancer Res 66, 10870-10877.

Perk, J., Iavarone, A., and Benezra, R. (2005). Id family of helix-loop-helix proteins in cancer. Nat Rev Cancer 5, 603-614.

Perk J. et al., Cancer Res 66, 10870 (Nov. 15, 2006).

S. S. Perry, Y. Zhao, L. Nie et al., Blood 110 (7), 2351 (2007).

Petreanu, L., and Alvarez-Buylla, A. (2002). Maturation and death of adult-born olfactory bulb granule neurons: role of olfaction. J Neurosci 22, 6106-6113.

Pollard, S. M., Conti, L., Sun, Y., Goffredo, D., and Smith, A. (2006). Adherent neural stem (NS) cells from fetal and adult forebrain. Cereb Cortex 16 Suppl 1, i112-120.

Ramon y Cajal, S. (1913-14). Estudios sobre la degeneracion y regeneracion del sistema nervioso. Madrid, Moya.

Reumers, V., Deroose, C. M., Krylyshkina, O., Nuyts, J., Geraerts, M., Mortelmans, L., Gijsbers, R., Van den Haute, C., Debyser, Z., and Baekelandt, V. (2008). Noninvasive and quantitative monitoring of adult neuronal stem cell migration in mouse brain using bioluminescence imaging. Stem cells (Dayton, Ohio) 26, 2382-2390.

Shen, Q., Wang, Y., Kokovay, E., Lin, G., Chuang, S. M., Goderie, S. K., Roysam, B., and Temple, S. (2008). Adult SVZ stem cells lie in a vascular niche: a quantitative analysis of niche cell-cell interactions. Cell Stem Cell 3, 289-300.

Spassky, N., Merkle, F. T., Flames, N., Tramontin, A. D., Garcia-Verdugo, J. M., and Alvarez-Buylla, A. (2005). Adult ependymal cells are postmitotic and are derived from radial glial cells during embryogenesis. J Neurosci 25, 10-18.

Tavazoie, M., Van der Veken, L., Silva-Vargas, V., Louissaint, M., Colonna, L., Zaidi, B., Garcia-Verdugo, J. M., and Doetsch, F. (2008). A specialized vascular niche for adult neural stem cells. Cell Stem Cell 3, 279-288.

van Praag, H., Schinder, A. F., Christie, B. R., Toni, N., Palmer, T. D., and Gage, F. H. (2002). Functional neurogenesis in the adult hippocampus. Nature 415, 1030-1034.

S. Warming, N. Costantino, D. L. Court et al., Nucleic Acids Res 33 (4), e36 (2005).

G. J. Woodhead, C. A. Mutch, E. C. Olson et al., J Eurosci 26 (48), 12620 (2006).

Ying, Q. L., Nichols, J., Chambers, I., and Smith, A. (2003). BMP induction of Id proteins suppresses differentiation and sustains embryonic stem cell self-renewal in collaboration with STAT3. Cell 115, 281-292.

What is claimed is:

1. A method for identifying cells as potential stem cells comprising the step of screening the cells for expression of Inhibitor of DNA Binding-1 (Id-1), wherein expression of Id1 is indicative that the cells are adult stem cells.

2. The method of claim 1, wherein the adult stem cells derived from neuronal tissue, olfactory epithelium, colon, bone marrow, breast epithelium, eye, or germline tissue.

3. The method of claim 1, wherein the cell are human.

4. The method of claim 2, wherein the screening step is performed using an oligonucleotide probe that hybridizes with Id1-encoding DNA or RNA.

5. The method of claim 2, wherein the screening step is performed using an Id1-specific antibody.

6. The method of claim 1, further comprising an additional screening step, performed before or after screening the cells for expression of Id1, said additional screening step comprising screening cells for expression of glial fibrillary acidic protein (GFAP).

7. A method for isolating stem cells from a sample, comprising the steps of:
combining a mixture of cells from which stem cells are to be isolated with a molecule that binds specifically to Inhibitor of DNA Binding-1 (Id-1), and
separating cells that bind to the molecule from cells that do not.

8. The method of claim 7, wherein in the separating step, cells that bind to the molecule are bound, via the molecule, to a solid support.

9. The method of claim 8, wherein the molecule is an antibody that binds to Id1.

10. The method of claim 7, wherein the separating step is performed using a fluorescence-activated cell sorter.

11. The method of claim 7, wherein the separating step is performed using a magnetic cell sorting technique.

12. The method of claim 7, wherein the mixture of cells comprises cells derived from neuronal tissue, olfactory epithelium, colon, bone marrow, breast epithelium, eye, or gernline tissue.

13. The method of claim 7, wherein the mixture of cells comprises human cells.

14. The method of claim 7, further comprising an additional isolation step, said second isolation step comprising combining the mixture of cells with a molecule that binds to glial fibrillary acidic protein (GFAP) and separating cells that bind to the molecule from cells that do not.

15. The method of claim 14, further comprising pre-separation enrichment step in which cells that express PSA-NCAM and/or NeuN are removed from the mixture of cells.

16. The method of claim 7, wherein the mixture of cells is identified as containing potential step cells by a method comprising the step of screening the mixture of cells for cells that express Id-1, wherein expression of Id1 is indicative that the cells are adult stem cells.

17. The method of claim 16, wherein the screening step is performed using an oligonucleotide probe that hybridizes with Id1-encoding DNA or RNA.

18. The method of claim 17, wherein the screening step is performed using an Id1-specific antibody.

19. The method of claim 16, wherein the separating step is performed using a fluorescence-activated cell sorter.

20. The method of claim 16, wherein the separating step is performed using a magnetic cell sorting technique.

21. The method of claim 16, wherein the mixture of cells comprises cells derived from neuronal tissue, olfactory epithelium, colon, bone marrow, breast epithelium, eye, or germline tissue.

22. The method of claim 16, wherein the mixture of cells comprises human cells.

23. The method of claim 16, further comprising an additional isolation step, said second isolation step comprising combining the mixture of cells with a molecule that binds to glial fibrillary acidic protein (GFAP) and separating cells that bind to the molecule from cells that do not.

24. The method of claim 23, further comprising pre-separation enrichment step in which cells that express PSA-NCAM and/or NeuN are removed from the mixture of cells.

25. The method of claim 16, further comprising pre-separation enrichment step in which cells that express PSA-NCAM and/or NeuN are removed from the mixture of cells.

26. The method of claim 7, further comprising pre-separation enrichment step in which cells that express PSA-NCAM and/or NeuN are removed from the mixture of cells.

27. A cell preparation comprising a plurality of cells in a liquid carrier, in which adult stem cells that express Id1 are enriched relative to a starting tissue extract.

28. The cell preparation according to claim 27, wherein the percentage of cells expressing Id1 is at least 2 fold the percentage of cells in the starting tissue extract.

29. The cell preparation of claim 27, wherein the percentage of cells expressing both Id1 and GFAP is enriched.

30. The cell preparation of claim 27, wherein the adult stem cells comprise B1 type adult neuronal stem cells.

31. The cell preparation of claim 30, wherein the cell preparation comprises at least 1% B1 type adult neuronal stem cells.

32. The cell preparation of claim 30, wherein the cell preparation comprises at least 2% B1 type adult neuronal stem cells.
